(19) United States
(12) Patent Application Publication
(10) Pub. No.: US 2008/0279803 A1
(43) Pub. Date: Nov. 13, 2008

(54) Method and Composition for Restructuring Keratin Fibers

(75) Inventors: Sabine Kainz, Moers (DE); Ursula Huchel, Koln (DE); Olaf Lammerschop, Krefeld (DE); Burkhard Muller, Hamburg (DE); Thorsten Knappe, Schenefeld (DE)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: HENKEL KOMMANDITGESELLSCHAFT AUF AKTIEN, Dusseldorf (DE)

(21) Appl. No.: 11/569,355

(22) PCT Filed: Apr. 8, 2005
(86) PCT No.: PCT/EP05/03697

§ 371 Date: Nov. 17, 2006

(30) Foreign Application Priority Data
May 18, 2004 (DE) DE102004024509

(51) Int. Cl. A61K 8/84; A61Q 5/04

(52) U.S. Cl. 424/70.5

(57) ABSTRACT

The invention relates to a method for restructuring keratin fibers, whereby a keratin fiber is contacted with (a) an aqueous preparation of at least one polysulfide of formula (I), wherein R<3> represents hydroxy or the group (II), R<4> represents hydrogen or the group (III), A is a bond or a bivalent saturated or mono- or polyunsaturated aliphatic or aromatic hydrocarbon group having 1 to 20 carbon atoms, which can be substituted by one or more halogen, hydroxy or carboxy groups and whereby the shortest bond between the carbonyl groups adjacent to group A consists of up to 12 carbon atoms, and wherein R<1> and R<2> are the same or different and are selected from R<5>O— and (IV), wherein R<5> represents hydrogen or an alkyl group having 1 to 6 carbon atoms, n is an integer of from 1 to 100, and whereby one or more carboxyl groups may be present in the form of one or more of its/their salts, and, either simultaneously or successively, a reducing agent, and then (b) with an oxidant. The invention also relates to preparations for use in the inventive methods.

The invention relates to a method for restructuring keratin fibers, in which a keratin fiber is brought into contact with an aqueous preparation of at least one polysulfide and a reducing agent simultaneously or successively, and then with an oxidizing agent. In addition, the invention relates to preparations for use in this method.

Keratin fibers, in particular hair, being a firm constituent of the human body and being an essential constituent of human clothing and home textiles, is of important significance in everyday life. Treatment with washing, cleaning, styling and coloring products for cleaning and shaping purposes, and their exposure to environmental influences, such as ozone, salt water and chlorinated water, IR, UV and thermal radiation (blow-drying) lead, over the course of time, to cumulative damage of the fibers and thus to a reduction in their quality. For example, both the cleaning of hair with shampoos, and also decorative shaping of the hairstyle through coloring or permanent waving are interventions which influence the natural structure and the properties of the hair. Consequently, after such a treatment, the wet and dry combability, hold, fullness, shine and tactility of the hair, for example, may be unsatisfactory. In the case of colored hair, particularly in the case of frequent hair washing, the hold of the color on the hair may also be unsatisfactory, leading to gradual fading of the color.

Not least as a result of the severe stressing of the hair, for example through coloring or permanent waving, and also as a result of cleaning the hair with shampoos and as a result of environmental stresses, the importance of care products with a sufficiently strong effect which is as long-lasting as possible is increasing. Such care compositions influence the natural structure and the properties of the hair. Thus, following such treatments, the wet and dry combability of the hair, the hold and the fullness of the hair, for example, can be improved, or the hair may be protected against an increased number of split ends.

It has therefore been customary for a long time to subject the hair to a special after-treatment. Here, the hair is treated, mostly in the form of a rinse, with special active ingredients, for example quaternary ammonium salts or special polymers. As a result of this treatment, depending on the formulation, the combability, the hold and the fullness of the hair are improved and the number of split ends is reduced.

In general, the active ingredients available both for separate after-treatment compositions and for combination preparations preferably act on the surface of the hair. For example, haircare compositions are known which impart shine, hold, fullness or better wet or dry combabilities to the hair or prevent split ends. However, of equal importance to the outer appearance of the hair is the inner structural cohesion of the hair fibers, which can be greatly influenced particularly during oxidative and reductive processes such as coloring and permanent waving.

Keratin fibers are subject to particular stresses during shaping methods such as the permanent waving of hair. The permanent shaping of keratin fibers is usually carried out by mechanically shaping the fibers and fixing the shape using suitable auxiliary means. Before and/or after this shaping, the fibers are treated with an aqueous preparation of a keratin-reducing substance and, after a contact time, rinsed with water or an aqueous solution. In a second step, the fibers are then treated with the aqueous preparation of an oxidizing agent. After a contact time, this too is rinsed out and the fibers are freed from the mechanical shaping means (curlers, papillotes).

The aqueous preparation of the keratin reducing agent is usually rendered alkaline so that, firstly, a sufficient fraction of the thiol functions is present in deprotonated form and, secondly, the fiber swells and, in so doing, allows the keratin-reducing substance to penetrate deeply into the fiber. The keratin-reducing substance cleaves some of the disulfide bonds in the keratin to —SH groups, resulting in a loosening of the peptide crosslinking and, as a result of the tension in the fibers due to mechanical shaping, in a new orientation of the keratin structure. Under the influence of the oxidizing agent, disulfide bonds are again joined, and, in this way, the keratin structure is newly fixed in the pregiven shape.

However, a negative side-effect of the permanent waving of hair carried out in this way is often an embrittling and dulling of the hair. In addition, in many cases, other properties such as wet and dry combability, feel, suppleness, softness, shine and tear strength are also influenced in an undesired way.

Care additives and film formers are often added to the permanent waving composition without, however, significantly improving the hair structure. For this, for example, high molecular weight polymers are used which attach to the uppermost layer of skin and hair, where they produce an external, subjectively perceptibly improved feel of the hair. Structural damage inside the hair, which is caused in the case of permanent waving primarily as a result of the reduction process cannot, however, be thereby reduced since the substances are not able, on account of their size, to penetrate into the hair.

EP 723 772 describes that greater swelling of the hair takes place as a result of alkalinizing agents such as basic amino acids together with cationic polymers in the waving composition. On the one hand, it leads to greater shaping and longer durability of the permanent wave, but on the other hand also to hair damage.

Laid-open specification GB 216 041 9 describes a method in which hair is firstly treated with a reducing agent which is then rinsed out. An aqueous protein hydrolyzate, preferably with a molecular weight greater than 50 000 daltons, is then applied to the areas to be treated, after which, finally, neutralization takes place. Although the area treated in this way did feel subjectively better, a reduction in hair damage on the inside did not take place.

The object of the present invention was to provide an improved method for restructuring keratin fibers which has advantages over the prior art and permits adequate effectiveness and action time. In addition, it should be possible to carry out the method under fiber-friendly conditions and to perform it, for example, in the course of a customary permanent waving treatment or a customary hair-straightening method.

The object according to the invention is achieved by a method in which keratin fibers are brought into contact with certain polysulfides and, simultaneously or subsequently, with a reducing agent and, in a further step, with an oxidizing agent.

The invention therefore firstly provides a method for restructuring keratin fibers in which a keratin fiber is brought into contact
(a) with an aqueous preparation of at least one polysulfide of the formula (I)

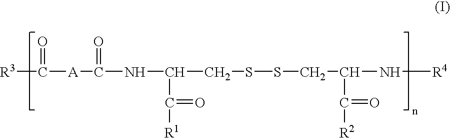

in which
R³ is hydroxy or the radical

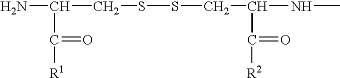

R⁴ is hydrogen or the radical

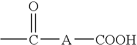

A is a bond or a divalent saturated or a mono- or polyunsaturated aliphatic or aromatic hydrocarbon radical having 1 to 20 carbon atoms, which may be substituted by one or more halogen, hydroxy or carboxy groups and where the shortest bond between the two carbonyl groups adjacent to group A consists of up to 12 carbon atoms, and in which
R¹ and R² may be identical or different and are chosen from
   R⁵O— and

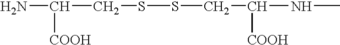

where R⁵ is hydrogen or an alkyl group having 1 to 6 carbon atoms,
n is an integer from 1 to 100,
and where one or more carboxyl group(s) may be in the form of one or more of its/their salt(s),
and a reducing agent,
simultaneously or successively, and then
(b) with an oxidizing agent.

Keratin fibers which can be used are, in principle, all animal hair, such as, for example, wool, horse hair, angora hair, furs, feathers, and silk and products or textiles produced therefrom. However, the method according to the invention is preferably suitable for the shaping of human hair and wigs produced therefrom. On account of the gentle processing conditions, it is particularly suitable for shaping the hair on the living body, i.e., for example, in connection with producing permanently waved hair or straightening curly hair.

Surprisingly, it has now been found that by using the method according to the invention it is possible to change the internal and external structure of keratin fibers in an advantageous way, i.e. in other words that restructuring of keratin fibers is made possible. For the purposes of the present invention, restructuring is understood as meaning in particular fiber strengthening, an increase in breaking force and/or a reduction in the damage to keratin fibers caused by a very wide variety of influences. Here, for example, the restoration of the natural strength plays a significant role. Restructured fibers can, for example, be characterized by increased breaking force, increased strength, increased elasticity and/or increased volume, which may be evident, for example, from greater fullness in a hairstyle. In addition, they can have improved shine, improved feel and/or be easier to comb.

The method according to the invention serves for the strengthening, protection and repair of keratin fibers and is very particularly suited to improving the hair structure and/or strengthening human hair. In particular, fiber properties such as strength, elasticity or volume are positively influenced in the sense of an increase in these properties. Furthermore, the method is suitable for styling purposes, such as imparting shape and retaining shape, and also for increasing color fastness, particularly the wash fastness of colored keratin fibers, in particular colored human hair. Wash fastness is to be understood as meaning the retention of the color of a colored keratin fiber with regard to color nuance and/or color intensity when the colored fiber is exposed to the effect of aqueous compositions, in particular surfactant-containing compositions such as shampoos. The method according to the invention is also suitable for protecting fibers against the harmful effect of light.

The method according to the invention requires no toxicologically unacceptable substances such as, for example, free-radical formers or intermediate free radicals.

In preferred embodiments of the invention, polysulfides are used in which, in formula (I), A is a divalent saturated aliphatic hydrocarbon radical having 2 to 6 carbon atoms, in particular the ethane-1,2-diyl radical.

It is also preferred if, in formula (I), n is an integer from 1 to 10.

Ways of producing polysulfides of the formula (I) suitable according to the invention are described in the patent U.S. Pat. No. 5,646,239 (in particular Examples 1 to 3), to which reference is hereby expressly made.

The polysulfides of the formula (I) used in the method according to the invention can be produced in a preferred way by polycondensing cystine or a cystine derivative in which the carboxyl groups are esterified or derivatized in some other way with a dicarbonyl compound. A suitable dicarbonyl compound is, in particular, a dicarboxylic acid derivative in which the carboxyl groups are present in activated form, such as, for example, a dicarbonyl chloride.

It is particularly preferred if, in the method according to the invention, the polysulfide of the formula (I) is used in the form of a condensation product which is obtainable by reacting cystine with a dicarboxylic acid derivative in which the carboxyl groups are present in activated form, such as, for example, a dicarbonyl dichloride. This reaction can be carried out, in a particularly advantageous embodiment, as interface condensation in a two-phase system.

The reducing agents which can be used in step (a) of the method according to the invention are preferably chosen from keratin-reducing compounds, in particular compounds with at least one thiol group, and derivatives thereof, and also from sulfites, hydrogensulfites and disulfites.

Compounds with at least one thiol group and derivatives thereof are, for example, thioglycolic acid, thiolactic acid, thiomalic acid, phenylthioglycolic acid, mercaptoethanesulfonic acid, and salts and esters thereof (such as, for example, isooctyl thioglycolate and isopropyl thioglycolate), cysteamine, cysteine, Bunte salts and salts of sulfurous acid. The monoethanolammonium salts or ammonium salts of thioglycolic acid and/or of thiolactic acid, and the respective free acids are preferably suitable. Within the scope of the method according to the invention, these are preferably used in the form of compositions which contain concentrations of from 0.5 to 2.0 mol/kg of these compounds and have a pH of from 5 to 12, in particular from 7 to 9.5. To establish these pHs, alkalinizing agents such as ammonia, alkali metal and ammonium carbonates and hydrogencarbonates or organic amines, such as monoethanolamine, are preferably used.

Examples of keratin-reducing compounds of the disulfite type are alkali metal disulfites, such as, for example, sodium disulfite (Na₂S₂O₅) and potassium disulfite (K₂S₂O₅), and magnesium disulfite and ammonium disulfite ((NH₄)₂S₂O₅). Ammonium disulfite may be preferred here according to the invention. Examples of keratin-reducing compounds of the hydrogensulfite type are hydrogensulfites as alkali metal, magnesium, ammonium or alkanolammonium salt based on a C₂-C₄-mono-, di- or trialkanolamine. Ammonium hydrogensulfite may here be a particularly preferred hydrogensulfite. Examples of keratin-reducing compounds of the sulfite type are sulfites as alkali metal, ammonium or alkanolammonium salt based on a C₂-C₄-mono-, di- or trialkanolamine. Ammonium sulfite is preferred here. Within the scope of the method according to the invention, the use of sulfite and/or disulfite and/or hydrogen sulfite takes place preferably at pH 5 to 8, in particular from pH 6 to 7.5. Preferred C₂-C₄-alkanolamines according to the invention are 2-aminoethanol (monoethanolamine) and N,N,N-tris(2-hydroxy-ethyl)amine (triethanolamine). Monoethanolamine is a particularly preferred C₂-C₄-alkanolamine which, for the purposes of the method according to the invention, is preferably used in the form of compositions which have a concentration of from 0.2 to 6% by weight of this amine, based on the total composition.

Reducing agents particularly preferred according to the invention are thioglycolic acid and thiolactic acid, and salts thereof.

Within the scope of the method according to the invention, the reducing agent is preferably used in the form of a composition which comprises the reducing agent in an amount of from 5 to 20% by weight, based on the total composition.

The temperature when bringing the reducing agent into contact with the fiber is preferably in a range from about 10 to about 60° C.

Suitable oxidizing agents which can be used in step (b) of the method according to the invention are preferably substances which are chosen from oxygen, air, H₂O₂, disulfides, sodium perborate and hydrates thereof, sodium and potassium bromate, sodium chlorite, sodium or potassium persulfate, sodium iodate, calcium or magnesium bromate, tetrathionates, glyoxal, glutaraldehyde, and mixtures of these substances. In the case of oxygen or of air as oxidizing agent, it may be advantageous if, in addition, catalytic amounts of manganese sulfate or cobalt sulfate or terpene derivatives are present.

A particularly preferred oxidizing agent is air and/or H₂O₂.

Within the scope of the method according to the invention, the oxidizing agents are preferably used in the form of aqueous preparations.

In a very particularly preferred embodiment of the invention, in step (a) of the method according to the invention, the reducing agent used is thioglycolic acid and/or thiolactic acid or one of their salts and, in step (b) of the method according to the invention, the oxidizing agent used is H₂O₂.

The at least one polysulfide of the formula (I) according to the invention is present in the preparation used in step (a) of the method according to the invention in an amount of in total 0.01 to 5% by weight, but in particular 0.1 to 2% by weight, based on the total weight of the preparation.

The preparation used in step (a) of the method according to the invention preferably has a pH between 6 and 10, in particular between 7 and 9.5. To adjust this pH, the preparations according to the invention can comprise alkalinizing agents, such as ammonia, alkali metal and ammoniumcarbonates and hydrogencarbonates or organic amines such as monoethanolamine.

In a further particularly preferred embodiment of the invention, in step (a) of the method according to the invention, cystine is also present besides the aqueous preparation of the polysulfide. Here, it is preferred if the cystine, based on the weight of the at least one polysulfide of the formula (I), is present in an amount of from 5 to 1000% by weight, but in particular 50 to 500% by weight.

In step (a) of the method according to the invention, it may be advantageous if succinic acid is also present besides the aqueous preparation of the polysulfide and optionally cystine.

Within the scope of the method according to the invention, the keratin fiber is brought into contact with the polysulfide and a reducing agent simultaneously or successively.

It is preferred if, in step (a) of the method, the fiber is brought into contact simultaneously with the polysulfide of the formula (I) and the reducing agent.

In a further preferred embodiment of the method, polysulfide and reducing agent are mixed together prior to application to the hair, for example 15 seconds to 12 hours prior to application to the hair. Depending on how the method is to be carried out, it may be preferred to mix the two components together shortly prior to application to the hair, for example 15 seconds to 15 minutes prior to application to the hair. However, it may also be advantageous if the two components are mixed together a relatively long time prior to application to the hair, for example 1 to 12 hours beforehand.

In step (a) of the method, the fiber remains in contact with the polysulfide of the formula (I) and/or the reducing agent preferably for a contact time from 1 to 60 minutes, but in particular from 5 to 30 minutes.

The fiber can be brought into contact with the polysulfide of the formula (I) and/or the reducing agent in step (a) of the method according to the invention in such a way that, besides the aqueous preparation of the polysulfide and/or the reducing agent, further substances are present. These substances are preferably chosen so that they form a carrier for the polysulfide and/or for the reducing agent that is suitable for the treatment of the fibers.

In a preferred embodiment of the invention, step (a) of the method according to the invention is carried out such that a fiber is brought into contact with a preparation (R) which comprises a polysulfide of the formula (I) and a reducing agent.

If the fiber is a hair, the substances present in the preparation (R) besides the polysulfide of the formula (I) preferably form a composition of the type which is familiar to the person skilled in the art of hair cosmetics as “waving composition”.

In a further preferred embodiment of the invention, in step (a) of the method according to the invention, the fiber is brought into contact firstly with a preparation (V) which comprises a polysulfide of the formula (I), and then with a reducing agent.

In a further embodiment of this process variant, the preparation (V) comprises some of the reducing agent used overall in the method according to the invention.

In a further embodiment of the invention, in step (a) of the method according to the invention, the fiber is brought into contact firstly with a reducing agent, and then with a preparation (Z) which comprises a polysulfide of the formula (I).

In a further embodiment of this process variant, the preparation (Z) comprises some of the reducing agent used overall in the method according to the invention.

The fiber can be brought into contact with the oxidizing agent in step (b) of the method according to the invention in such a way that further substances are present besides the oxidizing agent. These substances are preferably chosen so that they form a carrier for the oxidizing agent that is suitable for the treatment of the fiber.

In a preferred embodiment of the invention, step (b) of the method according to the invention is thus carried out by bringing a fiber into contact with a preparation (O) which comprises the oxidizing agent.

If the fiber is a hair, the preparation (O) preferably forms a composition of the type which is familiar to the person skilled in the art of hair cosmetics as “neutralizer”.

Between step (a) and step (b) of the method according to the invention, at least one intermediate step (c) can additionally take place, in which the fiber is treated and, in particular, rinsed with water or an aqueous preparation (C), which, in a preferred embodiment of the invention, is a composition of the type which is familiar to the person skilled in the art of hair cosmetics as “intermediate rinse” suitable for use in a permanent waving method. Such an intermediate rinse can, for example, comprise a care substance, e.g. a protein hydrolyzate, in a carrier.

Preferably, the fiber is rinsed with water between step (a) and step (b) of the method according to the invention.

In one of the preferred embodiments, the method according to the invention serves for the permanent shaping of keratin fibers, in particular human hair.

The preparations used in the method according to the invention can be solid, liquid, gel-like or pasty. They are preferably chosen from aqueous systems, natural or synthetic oils, water-in-oil or oil-in-water emulsions. Such systems and methods for their production are known in the prior art, to which reference is hereby made. The preparations can be formulated as cream, gel or liquid. In addition, it is possible to formulate the compositions in the form of foam aerosols which are bottled with a liquefied gas such as, for example, propane/butane mixtures, nitrogen, CO₂, air, NO₂, dimethyl ether, chlorofluorocarbon propellants or mixtures thereof in aerosol containers with foam valve. Preferably, the individual components of the method according to the invention are used as cream, gel or liquid. In addition, the preparations used according to the invention can be two-phase or multiphase. Two-phase and multiphase systems are systems in which there are at least two separate continuous phases. For example, in such systems, one aqueous phase and one or more, e.g. two, immiscible, nonaqueous phases, may be present separately from one another. Likewise possible are, for example, a water-in-oil emulsion and an aqueous phase present separately therefrom, or a water-in-oil emulsion and an aqueous phase present separately therefrom.

Furthermore, the invention provides an aqueous preparation for use in the method according to the invention which comprises at least one polysulfide of the formula (I).

The aqueous preparation preferably comprises 0.01 to 5% by weight, but in particular 0.1 to 2% by weight, of one or more polysulfides of the formula (I), based on the total weight of the preparation.

In a preferred embodiment of the invention, the preparation also comprises buffer substances, for example a combination of ammonia and ammonium hydrogencarbonate, which keep the pH of the preparation in a range from 7 to 9.5.

In a further preferred embodiment of the invention, the preparation further comprises cystine, for example 0.001 to 10% by weight, but in particular 0.05 to 5% by weight, of cystine, based on the total weight of the preparation.

In a further, likewise preferred embodiment of the invention, the preparation further comprises a keratin-reducing compound, in particular thioglycolic acid and/or thiolactic acid or a salt thereof.

As further constituents of the preparations used in the method according to the invention, active substances such as, for example, surfactants, complexing agents, polyols, fatty substances, oil bodies, polymers, protein hydrolyzates, amino acids, vitamins, plant extracts, hydroxycarboxylic acids, emulsifiers, penetration auxiliaries and silicone oils, can be used advantageously.

Suitable surfactants are surface-active substances from the group of anionic, amphoteric, zwitterionic and nonionic surfactants. The surfactants have, inter alia, the task of promoting wetting of the keratin surface by the treatment solution.

Suitable anionic surfactants are in principle all anionic surface-active substances, in particular those suitable for use on the human body. These are characterized by a solubilizing anionic group, such as, for example, a carboxylate, sulfate, sulfonate or phosphate group, and a lipophilic alkyl group with about 8 to 30 carbon atoms. Additionally, glycol or polyglycol ether groups, ester groups, ether groups and amide groups and also hydroxyl groups may be present in the molecule. Examples of suitable anionic surfactants are, in each case in the form of the sodium, potassium and ammonium and also the mono-, di- and trialkanolammonium salts having 2 to 4 carbon atoms in the alkanol group,
linear and branched fatty acids having 8 to 30 carbon atoms (soaps),
ether carboxylic acids of the formula R—O—(CH₂—CH₂O)ₓ—CH₂—COOH, in which R is a linear alkyl group having 8 to 30 carbon atoms and x=0 or 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic mono- and dialkyl esters having 8 to 24 carbon atoms in the alkyl group and sulfosuccinic monoalkyl polyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkylpolyglycol ether sulfates of the formula R—O(CH₂—CH₂O)ₓ—OSO₃H, in which R is a preferably linear alkyl group having 8 to 30 carbon atoms and x=0 or 1 to 12,
mixtures of surface-active hydroxysulfonates as in DE-A-37 25 030,
sulfated hydroxyalkyl polyethylene and/or hydroxyalkylene propylene glycol ethers as in DE-A-37 23 354,
sulfonates of unsaturated fatty acids having 8 to 24 carbon atoms and 1 to 6 double bonds as in DE-A-39 26 344,
esters of tartaric acid and citric acid with alcohols, which constitute addition products of about 2-15 molecules of ethylene oxide and/or propylene oxide onto fatty alcohols having 8 to 22 carbon atoms,
alkyl and/or alkenyl ether phosphates of the formula (E1-I),

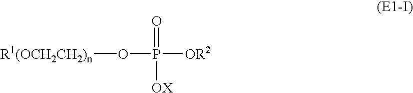

in which R¹ is preferably an aliphatic hydrocarbon radical having 8 to 30 carbon atoms, R² is hydrogen, a radical (CH₂CH₂O)ₙR¹ or X, n is numbers from 1 to 10 and X is hydrogen, an alkali metal or alkaline earth metal or NR³R⁴R⁵R⁶, where R³ to R⁶, independently of one another, are hydrogen or a C1 to C4-hydrocarbon radical,
sulfated fatty acid alkylene glycol esters of the formula (E1-II)

R⁷CO(AlkO)ₙSO₃M (E1-II)
in which R⁷CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation, as described in DE-A-197 36 906.5,
monoglyceride sulfates and monoglyceride ether sulfates of the formula (E1-III)

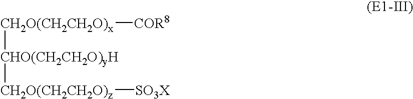

in which R⁸CO is a linear or branched acyl radical having 6 to 22 carbon atoms, x, y and z are in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid in the form of its sodium salts. Preference is given to using monoglyceride sulfates of the formula (E1-III) in which R⁸CO is a linear acyl radical having 8 to 18 carbon atoms, as have been described, for example, in EP-B1 0 561 825, EP-B1 0 561 999, DE-A1 42 04 700 or by A. K. Biswas et al. in J. Am. Oil. Chem. Soc. 37, 171 (1960) and F. U. Ahmed in J. Am. Oil. Chem. Soc. 67, 8 (1990),
amide ether carboxylic acids, as described in EP 0 690 044,
condensation products of C₈-C₃₀-fatty alcohols with protein hydrolyzates and/or amino acids and derivatives thereof, which are known to the person skilled in the art as protein fatty acid condensates, such as, for example, the Lamepon® grades, Gluadin® grades, Hostapon® KCG or the Amisoft® grades.

Preferred anionic surfactants are alkylsulfates, alkyl polyglycol ether sulfates and ether carboxylic acids having 10 to 18 carbon atoms in the alkyl group and up to 12 glycol ether groups in the molecule, sulfosuccinic mono- and dialkyl esters having 8 to 18 carbon atoms in the alkyl group and sulfosuccinic monoalkylpolyoxyethyl esters having 8 to 18 carbon atoms in the alkyl group and 1 to 6 oxyethyl groups, monoglyceride sulfates, alkyl and alkenyl ether phosphates, and protein fatty acid condensates.

Suitable cationic surfactants are in principle all cationic surface-active substances, in particular surfactants of the quaternary ammonium compound type, the ester quat type and the amidoamine type. Preferred quaternary ammonium compounds are ammonium halides, in particular chlorides and bromides, such as alkyltrimethylammonium chlorides, dialkyldimethylammonium chlorides and trialkylmethylammonium chlorides, e.g. cetyltrimethylammonium chloride and bromide, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the abovementioned surfactants preferably have 10 to 18 carbon atoms.

Ester quats are known substances which contain both at least one ester function and also at least one quaternary ammonium group as structural element. Preferred ester quats are quaternized ester salts of fatty acids with triethanolamine, quaternized ester salts of fatty acids with diethanol-alkylamines and quaternized ester salts of fatty acids with 1,2-dihydroxy-propyldialkylamines. Such products are sold, for example, under the trade names Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are usually prepared by amidation of natural or synthetic fatty acids and fatty acid cuts with dialkylaminoamines. One compound from this group of substances which is particularly suitable according to the invention is the stearamidopropyldimethylamine commercially available under the name Tegoamid® S18.

The cationic surfactants are present in the preparations used according to the invention preferably in amounts of from 0.05 to 10% by weight, based on the overall preparation. Amounts of from 0.1 to 5% by weight are particularly preferred.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyl dimethylammonium glycinate, N-acylaminopropyl-N,N-dimethylammonium glycinates, for example cocoacyl aminopropyldimethylammonium glycinate, and 2-alkyl-3-carboxymethyl-3-hydroxyethylimidazolines having in each case 8 to 18 carbon atoms in the alkyl or acyl group, and cocoacyl aminoethyl hydroxyethylcarboxymethyl glycinate. A preferred zwitterionic surfactant is the fatty acid amide derivative known under the INCI name Cocoamidopropyl Betaine.

Ampholytic surfactants are understood as meaning those surface-active compounds which, apart from a C₈-C₂₄-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 24 carbon atoms in the alkyl group. Particularly preferred ampholytic surfactants are N-cocoalkylaminopropionate, cocoacylaminoethylaminopropionate and C₁₂-C₁₈-acylsarcosine.

Nonionic surfactants comprise, as hydrophilic group, e.g. a polyol group, a polyalkylene glycol ether group or a combination of polyol and polyglycol ether group. Such compounds are, for example,
addition products of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group,
addition products, terminally capped with a methyl or C₂-C₆ alkyl radical, of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group, such as, for example, the grades obtainable under the trade names Dehydrol® LS, Dehydrol® LT (Cognis),
C₁₂-C₃₀-fatty acid mono- and diesters of addition products of from 1 to 30 mol of ethylene oxide onto glycerol,
addition products of from 5 to 60 mol of ethylene oxide onto castor oil and hydrogenated castor oil,
polyol fatty acid esters, such as, for example, the commercial product Hydagen® HSP (Cognis) or Sovermol grades (Cognis),
alkoxylated triglycerides,
alkoxylated fatty acid alkyl esters of the formula (E4-I)

R¹CO—(OCH₂CHR²)_{w}OR³ (E4-I)
in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20,
amine oxides,
hydroxyl mixed ethers, as are described, for example, in DE-A 19738866,
sorbitan fatty acid esters and addition products of ethylene oxide onto sorbitan fatty acid esters, such as, for example, the polysorbates,
sugar fatty acid esters and addition products of ethylene oxide onto sugar fatty acid esters,
addition products of ethylene oxide onto fatty acid alkanolamides and fatty amines,
sugar surfactants of the alkyl and alkenyl oligoglycoside type according to formula (E4-II),

R⁴O-[G]ₚ (E4-II)
in which R⁴ is an alkyl or alkenyl radical having 4 to 22 carbon atoms, G is a sugar radical having 5 or 6 carbon atoms and p is numbers from 1 to 10. They can be obtained by the relevant methods of preparative organic chemistry. As a representative of the extensive literature, reference may be made here to the overview paper by Biermann et al. in Starch/Stärke 45, 281 (1993), B. Salka in Cosm. Toil. 108, 89 (1993) and J. Kahre et al. in SÖFW-Journal Volume 8, 598 (1995).
The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in the general formula (E4-II) gives the degree of oligomerization (DP), i.e. the distribution of monoglycosides and oligoglycosides and is a number between 1 and 10. While p in the individual molecule must always be an integer and here can primarily assume the values p=1 to 6, the value p for a specific alkyl oligoglycoside is an analytically determined calculated value, which in most cases is a fraction. Preferably, alkyl and/or alkenyl oligoglycosides with an average degree of oligomerization p of from 1.1 to 3.0 are used. From the point of view of application, preference is given to those alkyl and/or alkenyl oligoglycosides whose degree of oligomerization is less than 1.7 and is in particular between 1.2 and 1.4. The alkyl or alkenyl radical R⁴ can be derived from primary alcohols having 4 to 11, preferably 8 to 10, carbon atoms. Typical examples are butanol, caproic alcohol, caprylic alcohol, capric alcohol and undecyl alcohol, and technical-grade mixtures thereof, as are obtained, for example, in the hydrogenation of technical-grade fatty acid methyl esters or in the course of the hydrogenation of aldehydes from the Roelen oxosynthesis. Preference is given to alkyl oligoglucosides of chain length C₈-C₁₀ (DP=1 to 3), which are produced as forerunning in the distillative separation of technical-grade C₈-C₁₈-coconut fatty alcohol and can be contaminated with a fraction of less than 6% by weight of C₁₂-alcohol, and also alkyl oligoglucosides based on technical-grade C_{9/11}-oxo alcohols (DP=1 to 3). In addition, the alkyl and alkenyl radical R¹⁵ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Preference is given to alkyl oligoglucosides based on hydrogenated C_{12/14}-coconut alcohol having a DP of from 1 to 3.
Sugar surfactants of the fatty acid N-alkylpolyhydroxyalkylamide type, a nonionic surfactant of the formula (E4-III),

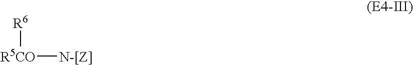

in which R⁵CO is an aliphatic acyl radical having 6 to 22 carbon atoms, R⁶ is hydrogen, an alkyl or hydroxyalkyl radical having 1 to 4 carbon atoms and [Z] is a linear or branched polyhydroxyalkyl radical having 3 to 12 carbon atoms and 3 to 10 hydroxyl groups. The fatty acid N-alkylpolyhydroxyalkylamides are known substances which can usually be obtained by reductive amination of a reducing sugar with ammonia, an alkylamine or an alkanolamine and subsequent acylation with a fatty acid, a fatty acid alkyl ester or a fatty acid chloride. With regard to the method of preparation, reference may be made to the US patent specifications U.S. Pat. No. 1,985,424, U.S. Pat. No. 2,016,962 and U.S. Pat. No. 2,703,798, and the international patent application WO 92/06984. An overview of this topic by H. Kelkenberg is given in Tens. Surf. Det. 25, 8 (1988). The fatty acid N-alkylpolyhydroxyalkylamides are preferably derived from reducing sugars having 5 or 6 carbon atoms, in particular from glucose. The preferred fatty acid N-alkylpolyhydroxyalkylamides are therefore fatty acid N-alkylglucamides, as are given by the formula (E4-IV):

R⁷CO—NR⁸—CH₂—(CHOH)₄CH₂OH (E4-IV)
As fatty acid N-alkylpolyhydroxyalkylamides, preference is given to using glucamides of the formula (E4-IV) in which R⁸ is hydrogen or an alkyl group and R⁷CO is the acyl radical of caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, arachic acid, gadoleic acid, behenic acid or erucic acid or technical-grade mixtures thereof. Particular preference is given to fatty acid N-alkylglucamides of the formula (E4-IV) which are obtained by reductive amination of glucose with methylamine and subsequent acylation with lauric acid or C12/14-coconut fatty acid or a corresponding derivative. In addition, the polyhydroxyalkylamides can also be derived from maltose and palatinose.

Preferred nonionic surfactants have proven to be the alkylene oxide addition products onto saturated linear fatty alcohols and fatty acids having in each case 2 to 30 mol of ethylene oxide per mole of fatty alcohol or fatty acid, as well as fatty acid esters of ethoxylated glycerol.

These compounds are characterized by the following parameters. The alkyl radical R comprises 6 to 22 carbon atoms and can either be linear or branched. Preference is given to primary linear and 2-position methyl-branched aliphatic radicals. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. When using so-called oxo alcohols as starting materials, compounds with an uneven number of carbon atoms in the alkyl chain predominate.

Compounds with alkyl groups used as surfactant may in each case be uniform substances. However, it is generally preferred when preparing the substances to start from native vegetable or animal raw materials, thus resulting in mixtures of substances with various alkyl chain lengths which depend on the particular raw material.

In the case of the surfactants which represent addition products of ethylene oxide and/or propylene oxide onto fatty alcohols or derivatives or these addition products, it is possible to use either products with a “normal” homolog distribution and also those with a narrowed homolog distribution. “Normal” homolog distribution is understood here as meaning mixtures of homologs which are obtained during the reaction of fatty alcohol and alkylene oxide using alkali metals, alkali metal hydroxides or alkali metal alkoxides as catalysts. Narrowed homolog distributions, by contrast, are obtained if, for example, hydrotalcites, alkaline earth metal salts of ether carboxylic acids, alkaline earth metal oxides, hydroxides and alkoxides are used as catalysts. The use of products with a narrowed homolog distribution may be preferred.

Particularly preferred surfactants are protein-fatty acid condensates, cocoaamphodiacetates and fatty acid sulfates and ethylene oxide and/or propylene oxide adducts thereof.

Suitable complexing agents are, for example, EDTA, NTA, HEDP, organophosphonic acids, β-alaninediacetic acid, and dipicolinic acid or mixtures of these substances.

Suitable polyols are, for example, glycerol and partial glycerol ethers, 2-ethyl-1,3-hexanediol, 1,3-butanediol, 1,4-butanediol, 1,2-propanediol, 1,3-propanediol, pentanediols, for example 1,2-pentanediol, hexanediols, for example 1,2-hexanediol or 1,6-hexanediol, dodecanediol, in particular 1,2-dodecanediol, neopentyl glycol and ethylene glycol. In particular, 2-ethyl-1,3-hexanediol, 1,2-propanediol, 1,3-propanediol and 1,3-butanediol have proven to be particularly well suited.

These polyols are present in the preparations used according to the invention preferably in amounts of 1-10% by weight, in particular 2-10% by weight, based on the total preparation.

According to the invention, it is of course also possible to use alcohols with only limited miscibility with water, particularly if multiphase systems are to be obtained.

“Limited miscibility with water” is understood as meaning those alcohols which are soluble in water at 20° C. to not more than 10% by weight, based on the mass of water.

Further active substances which may be used are fatty substances. Fatty substances are to be understood as meaning fatty acids, fatty alcohols, natural and synthetic waxes, which may be present either in solid form or as liquid in aqueous dispersion, and natural and synthetic cosmetic oil components.

Fatty acids which can be used are linear and/or branched, saturated and/or unsaturated fatty acids having 6-30 carbon atoms in amounts of 0.1-15% by weight, based on the total composition. Fatty alcohols which can be used are saturated, mono- or polyunsaturated, branched or unbranched fatty alcohols having C₆-C₃₀ carbon atoms in amounts of 0.1-30% by weight, based on the total preparation.

Natural and synthetic cosmetic oil bodies which can be used according to the invention as active substances are, in particular:
vegetable oils. Examples of such oils are sunflower oil, olive oil, soya oil, rapeseed oil, almond oil, jojoba oil, orange oil, wheat germ oil, peach kernel oil and the liquid fractions of coconut oil. However, other triglyceride oils, such as the liquid fractions of beef tallow, and synthetic triglyceride oils are also suitable.
liquid paraffin oils, isoparaffin oils and synthetic hydrocarbons, and di-n-alkyl ethers having in total between 12 and 36 carbon atoms, in particular 12 and 24 carbon atoms, such as, for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, and di-tert-butyl ether, diisopentyl ether, di-3-ethyldecyl ether, tert-butyl n-octyl ether, isopentyl n-octyl ether and 2-methylpentyl n-octyl ether. The commercially available compounds 1,3-di(2-ethylhexyl)cyclohexane (Cetiol® S) and di-n-octyl ether (Cetiol® OE) may be preferred.

The amount of natural and synthetic cosmetic oil bodies used in the preparations used according to the invention is usually 0.1-30% by weight, based on the total preparation, preferably 0.1-20% by weight, and in particular 0.1-15% by weight.

The total amount of oil and fat components in the preparations according to the invention is usually 0.1-75% by weight, based on the total preparation. Amounts of 0.1-35% by weight are preferred according to the invention.

In addition, it has been found that polymers are advantageously used for the purposes of the method according to the invention. In a preferred embodiment, polymers are therefore added to the preparations used according to the invention, with both cationic, anionic, amphoteric and also nonionic polymers having proven to be effective.

Cationic polymers are to be understood as polymers which have, in the main chain and/or side chain, a group which may be “temporarily” or “permanently” cationic. According to the invention, “permanently cationic” is used to refer to those polymers which, irrespective of the pH of the preparation, have a cationic group. These are usually polymers which contain a quaternary nitrogen atom, for example in the form of an ammonium group. Preferred cationic groups are quaternary ammonium groups. In particular, those polymers in which the quaternary ammonium groups are bonded via a C1-4-hydrocarbon group to a polymer main chain constructed from acrylic acid, methacrylic acid or derivatives thereof have proven to be particularly suitable.

Homopolymers of the general formula (IX),

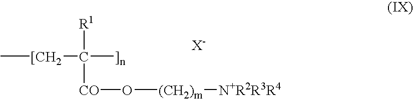

in which R¹═H or —CH₃, R², R³ and R⁴, independently of one another, are chosen from C1-4-alkyl, -alkenyl or -hydroxyalkyl groups, m=1, 2, 3 or 4, n is a natural number and X⁻ is a physiologically compatible organic or inorganic anion, and copolymers consisting essentially of the monomer units listed in formula (IX), and nonionogenic monomer units, are particularly preferred cationic polymers. Within the scope of these polymers, preference is given according to the invention to those for which at least one of the following conditions applies:
R¹ is a methyl group
R², R³ and R⁴ are methyl groups
m has the value 2.

Suitable physiologically compatible counterions X⁻ are, for example, halide ions, sulfate ions, phosphate ions, methosulfate ions, and organic ions, such as lactate, citrate, tartrate and acetate ions. Preference is given to halide ions, in particular chloride.

A particularly suitable homopolymer is the, if desired crosslinked, poly(methacryloyloxyethyltrimethylammonium chloride) with the INCI name Polyquaternium-37. Crosslinking can take place, if desired, with the help of polyolefinically unsaturated compounds, for example divinylbenzene, tetraallyloxyethane, methylenebisacrylamide, diallyl ether, polyallyl polyglyceryl ether, or allyl ethers of sugars or sugar derivatives, such as erythritol, pentaerythritol, arabitol, mannitol, sorbitol, sucrose or glucose. Methylenebisacrylamide is a preferred crosslinking agent. Further particularly suitable polymers are Polyquaternium-6, -7, -22 and -39, and Quaternium-52.

The homopolymer is preferably used in the form of a nonaqueous polymer dispersion which should have a polymer fraction not below 30% by weight. Such polymer dispersions are commercially available under the names Salcare® SC 95 (about 50% polymer fraction, further components: mineral oil (INCI name: Mineral Oil) and tridecyl polyoxypropylene polyoxyethylene ether (INCI name: PPG-1-Trideceth-6)) and Salcare® SC 96 (about 50% polymer fraction, further components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (IX) comprise, as nonionogenic monomer units, preferably acrylamide, methacrylamide, acrylic acid C₁₋₄-alkyl esters and methacrylic acid C₁₋₄-alkyl esters. Of these nonionogenic monomers, acrylamide is particularly preferred. As in the case of the homopolymers described above, these copolymers may also be crosslinked. A copolymer preferred according to the invention is the cross inked acrylamide-methacryloyloxyethyltrimethylammonium chloride copolymer. Such copolymers, in which the monomers are present in a weight ratio of about 20:80, are commercially available as about 50% strength nonaqueous polymer dispersion under the name Salcare® SC 92.

Further preferred cationic polymers are, for example,
quaternized cellulose derivatives, as are commercially available under the names Celquat® and Polymer JR®. The compounds Celquat® H 100, Celquat® L 200 and Polymer JR® 400 are preferred quaternized cellulose derivatives,
cationic alkyl polyglycosides as in DE-C 44 13 686,
cationized honey, for example the commercial product Honeyquat® 50,
cationic guar derivatives, such as, in particular, the products sold under the trade names Cosmedia® Guar and Jaguar®,
polysiloxanes with quaternary groups, such as, for example, the commercially available products Q2-7224 (manufacturer: Dow Corning; a stabilized trimethylsilylamodimethicone), Dow Corning® 929 emulsion (comprising a hydroxylamino-modified silicone, which is also referred to as amodimethicone), SM-2059 (manufacturer: General Electric), SLM-55067 (manufacturer: Wacker) and Abil®-Quat 3270 and 3272 (manufacturer: Th. Goldschmidt), diquaternary polydimethylsiloxanes, quaternium-80),
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are supplied under the names Luviquat® FC 370, FC 550, FC 905 and HM 552,
quaternized polyvinyl alcohol,
and the polymers with quaternary nitrogen atoms in the polymer main chain known under the names Polyquaternium 2, Polyquaternium 17, Polyquaternium 18 and Polyquaternium 27.

As cationic polymers, it is likewise possible to use the polymers known under the names Polyquaternium-24 (commercial product e.g. Quatrisoft® LM 200). According to the invention, it is likewise possible to use the copolymers of vinylpyrrolidone, as are available as commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Further cationic polymers which can be used according to the invention are the so-called temporarily cationic polymers. These polymers usually contain an amino group which, at certain pH values, is in the form of a quaternary ammonium group and thus cationic. Preference, for example, is given to chitosan and derivatives thereof, as are freely available commercially, for example, under the trade names Hydagen® CMF, Hydagen® HCMF, Kytamer® PC and Chitolam® NB/101.

Cationic polymers preferred according to the invention are cationic cellulose derivatives and chitosan and derivatives thereof, in particular the commercial products Polymer® JR 400, Hydagen® HCMF and Kytamer® PC, cationic guar derivatives, cationic honey derivatives, in particular the commercial product Honeyquat® 50, cationic alkyl polyglycosides as in DE-C 44 13 686 and polymers of the polyquaternium-37 type.

The anionic polymers which can be used in the preparations of the method according to the invention are anionic polymers which have carboxylate and/or sulfonate groups. Examples of anionic monomers of which such polymers can consist are acrylic acid, methacrylic acid, crotonic acid, maleic anhydride and 2-acrylamido-2-methylpropanesulfonic acid. Here, the acidic groups can be completely or partially in the form of the sodium, potassium, ammonium, mono- or triethanolammonium salt. Preferred monomers are 2-acrylamido-2-methylpropanesulfonic acid and acrylic acid.

Anionic polymers which have proven to be very particularly effective are those which contain 2-acrylamido-2-methylpropanesulfonic acid as the sole monomer or comonomer, where the sulfonic acid group can be completely or partially present as sodium, potassium, ammonium, mono- or triethanolammonium salt.

For example, such a homopolymer of 2-acrylamido-2-methylpropanesulfonic acid is commercially available under the name Rheothik® 11-80.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers, and in particular polyacrylamide copolymers with monomers containing sulfonic acid groups. A particularly preferred anionic copolymer consists of 70 to 55 mol % acrylamide and 30 to 45 mol % 2-acrylamido-2-methylpropanesulfonic acid, where the sulfonic acid group is present completely or partially as sodium, potassium, ammonium, mono- or triethanolammonium salt. This copolymer may also be in crosslinked form, in which case the crosslinking agents used are preferably polyolefinically unsaturated compounds such as tetraallyloxyethane, allylsucrose, allylpentaerythritol and methylenebisacrylamide.

Likewise preferred anionic homopolymers are uncrosslinked and crosslinked polyacrylic acids. Here, allyl ethers of pentaerythritol, of sucrose and of propylene may be preferred crosslinking agents. Such compounds are commercially available, for example, under the trade name Carbopol®.

Copolymers of maleic anhydride and methyl vinyl ether, in particular those with crosslinkings, are likewise highly suitable polymers. A maleic acid-methyl vinyl ether copolymer crosslinked with 1,9-decadienes is commercially available under the name Stabileze® QM.

In addition, polymers which can be used in all aqueous preparations of the method according to the invention are amphoteric polymers. The term amphoteric polymers includes both those polymers which contain both free amino groups and free —COOH or SO₃H groups in the molecule and are capable of forming internal salts, and also zwitterionic polymers which contain quaternary ammonium groups and —COO⁻ or —SO₃⁻ groups in the molecule, and those polymers which contain —COOH or SO₃H groups and quaternary ammonium groups.

One example of an amphopolymer which can be used according to the invention is the acrylic resin obtainable under the name Amphomer®, which is a copolymer of tert-butylaminoethyl methacrylate, N-(1,1,3,3-tetramethylbutyl)acrylamide and two or more monomers from the group acrylic acid, methacrylic acid and monoesters thereof.

Further amphoteric polymers which can be used according to the invention are the compounds specified in the British laid-open specification 2 104 091, the European laid-open specification 47 714, the European laid-open specification 217 274, the European laid-open specification 283 817 and the German laid-open specification 28 17 369.

Preferably used amphoteric polymers are those polymers which are essentially composed of

(a) monomers with quaternary ammonium groups of the general formula (X),

R¹—CH═CR²—CO-Z-(CₙH₂ₙ)—N⁽⁺⁾R³R⁴R⁵A⁽⁻⁾ (X)

in which R¹ and R², independently of one another, are hydrogen or a methyl group and R³, R⁴ and R⁵, independently of one another, are alkyl groups having 1 to 4 carbon atoms, Z is an NH group or an oxygen atom, n is an integer from 2 to 5 and A⁽⁻⁾ is the anion of an organic or inorganic acid, and
(b) monomeric carboxylic acids of the general formula (XI),

R⁶—CH═CR⁷—COOH (XI)

in which R⁶ and R⁷, independently of one another, are hydrogen or methyl groups.

These compounds can be used according to the invention either directly or in salt form, which is obtained by neutralization of the polymers, for example with an alkali metal hydroxide. As regards the details of the production of these polymers, reference is expressly made to the contents of German laid-open specification 39 29 973. Very particular preference is given to those polymers in which monomers of type (a) are used in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (a). The monomer (b) used for the specified polymers is preferably acrylic acid.

In addition, nonionogenic polymers may be present in all aqueous preparations of the method according to the invention.

Suitable nonionogenic polymers are, for example:
vinylpyrrolidone/vinyl ester copolymers, as are sold, for example, under the trade name Luviskol® (BASF). Luviskol® VA 64 and Luviskol® VA 73, each vinylpyrrolidone/vinyl acetate copolymers, are likewise preferred nonionic polymers.
cellulose ethers, such as hydroxypropylcellulose, hydroxyethylcellulose and methylhydroxypropylcellulose, as are sold, for example, under the trade names Culminal® and Benecel® (AQUALON).
shellac
polyvinylpyrrolidones, as are sold, for example, under the name Luviskol® (BASF).
siloxanes. These siloxanes may either be water-soluble or water-insoluble. Both volatile and nonvolatile siloxanes are suitable, with nonvolatile siloxanes being understood as meaning compounds whose boiling point at atmospheric pressure is above 200° C. Preferred siloxanes are polydialkylsiloxanes, such as, for example, polydimethylsiloxane, polyalkylarylsiloxanes, such as, for example, polyphenylmethylsiloxane, ethoxylated polydialkylsiloxanes, and polydialkylsiloxanes which contain amine and/or hydroxy groups, and cyclomethicones.
glycosidically substituted silicones as in EP 0612759 B1.

According to the invention, it is also possible for the preparations used to comprise two or more, in particular two, different polymers of equal charge and/or in each case one ionic polymer and one amphoteric and/or nonionic polymer.

The polymers are present in the preparations used according to the invention preferably in amounts of from 0.05 to 10% by weight, based on the total preparation. Amounts of from 0.1 to 5% by weight, in particular from 0.1 to 3% by weight, are particularly preferred.

In addition, protein hydrolyzates and/or amino acids and derivatives thereof may be present in the preparations used according to the invention. Protein hydrolyzates are product mixtures which are obtained by acidically, basically or enzymatically catalyzed degradation of proteins. According to the invention, the term protein hydrolyzates is also understood as meaning total hydrolyzates, and individual amino acids and derivatives thereof, and mixtures of different amino acids. In addition, according to the invention, polymers constructed from amino acids and amino acid derivatives are understood by the term protein hydrolyzates. The latter include, for example, polyalanine, polyasparagine, polyserine etc. Further examples of compounds which can be used according to the invention are L-alanyl-L-proline, polyglycine, glycyl-L-glutamine or D,L-methionine-S-methylsulfonium chloride. According to the invention, it is also of course possible to use β-amino acids and derivatives thereof, such as β-alanine, anthranilic acid or hippuric acid. The molecular weight of the protein hydrolyzates which can be used according to the invention is between 75, the molecular weight for glycine, and 200 000, preferably the molecular weight is 75 to 50 000 and very particularly preferably 75 to 20 000 daltons.

According to the invention, protein hydrolyzates both of vegetable origin, and also of animal or marine or synthetic origin may be used.

Animal protein hydrolyzates are, for example, the protein hydrolyzates of elastin, collagen, keratin, silk and milk protein, which may also be in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, the use of protein hydrolyzates of vegetable origin, e.g. soybean, almond, pea, potato and wheat protein hydrolyzates, is preferred. Such products are available, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although the use of protein hydrolyzates as such is preferred, instead of them it is in some cases also possible to use amino acid mixtures obtained in other ways. It is likewise possible to use derivatives of protein hydrolyzates, for example in the form of their fatty acid condensation products. Such products are sold, for example, under the names Lamepon® (Cognis), Lexein® (Inolex), Crolastin® (Croda) or Crotein® (Croda).

The protein hydrolyzates or derivatives thereof are present in the preparations used according to the invention preferably in amounts of from 0.1 to 10% by weight, based on the total preparation. Amounts of from 0.1 to 5% by weight are particularly preferred.

In addition, 2-pyrrolidinone-5-carboxylic acid and/or derivatives thereof can be used in the preparations of the method according to the invention. Preference is given to the sodium, potassium, calcium, magnesium or ammonium salts in which the ammonium ion carries one to three C₁- to C₄-alkyl groups besides hydrogen. The sodium salt is very particularly preferred. The amounts used in the preparations according to the invention are 0.05 to 10% by weight, based on the total preparation, particularly preferably 0.1 to 5% by weight, and in particular 0.1 to 3% by weight.

The use of vitamins, provitamins and vitamin precursors, and derivatives thereof has likewise proven advantageous.

In this connection, according to the invention, preference is given to those vitamins, provitamins and vitamin precursors which are usually assigned to the groups A, B, C, E, F and H.

The group of substances referred to as vitamin A includes retinol (vitamin A₁) and 3,4-didehydroretinol (vitamin A₂). β-Carotene is the provitamin of retinol. According to the invention, suitable vitamin A components are, for example, vitamin A acid and esters thereof, vitamin A aldehyde and vitamin A alcohol, and esters thereof, such as the palmitate and the acetate. The preparations used according to the invention comprise the vitamin A component preferably in amounts of 0.05-1% by weight, based on the total preparation.

The vitamin B group or the vitamin B complex includes, inter alia,
vitamin B₁ (thiamine)
vitamin B₂ (riboflavin)
vitamin B₃. This term often includes the compounds nicotinic acid and nicotinamide (niacinamide). According to the invention, preference is given to nicotinamide, which is present in the preparations used according to the invention preferably in amounts of from 0.05 to 1% by weight, based on the total preparation.
vitamin B₅ (pantothenic acid, panthenol and pantolactone). Within this group, preference is given to using panthenol and/or pantolactone. Derivatives of panthenol which can be used according to the invention are, in particular, the esters and ethers of panthenol, and cationically derivatized panthenols. Individual representatives are, for example, panthenol triacetate, panthenol monoethyl ether and its monoacetate, and the cationic panthenol derivatives disclosed in WO 92/13829. The specified compounds of the vitamin B₅ type are present in the preparations used according to the invention preferably in amounts of 0.05-10% by weight, based on the total preparation. Amounts of 0.1-5% by weight are particularly preferred.
vitamin B₆ (pyridoxine and pyridoxamine and pyridoxal),
vitamin C (ascorbic acid). Vitamin C is used in the preparations used according to the invention preferably in amounts of from 0.1 to 3% by weight, based on the total preparation. The use in the form of the palmitic acid ester, the glucosides or phosphates may be preferred. Use in combination with tocopherols may likewise be preferred.
vitamin E (tocopherols, in particular α-tocopherol). Tocopherol and its derivatives, which include in particular the esters such as the acetate, the nicotinate, the phosphate and the succinate, are present in the preparations used according to the invention preferably in amounts of 0.05-1% by weight, based on the total preparation.
vitamin F. The term “vitamin F” is usually understood as meaning essential fatty acids, in particular linoleic acid, linolenic acid and arachidonic acid.
vitamin H. Vitamin H refers to the compound (3aS,4S,6aR)-2-oxohexahydrothienol[3,4-d]imidazole-4-valeric acid, although the trivial name Biotin has caught on in the meantime. Biotin is present in the preparations used according to the invention preferably in amounts of from 0.0001 to 1.0% by weight, in particular in amounts of from 0.001 to 0.01% by weight.

Preferably, the preparations used according to the invention comprise vitamins, provitamins and vitamin precursors from the groups A, B, E and H.

Panthenol, pantolactone, pyridoxine and its derivatives, and nicotinamide and biotin are particularly preferred.

Finally, plant extracts may be used in the preparations of the method according to the invention.

Usually, these extracts are produced by extraction of the whole plant. However, in individual cases, it may also be preferred to produce the extracts exclusively from flowers and/or leaves of the plant.

With regard to the plant extracts which can be used according to the invention, reference is made in particular to the extracts which are listed in the table starting on page 44 of the 3rd edition of the Introduction to the Ingredients Declaration of Cosmetic Compositions, published by the Industrieverband Körperpflege und Waschmittel e.V. (IKW), Frankfurt.

According to the invention, the extracts from green tea, oak bark, stinging nettle, hamamelis, hops, henna, chamomile, burdock, horsetail, hawthorn, linden blossom, almond, aloe vera, fir needle, horsechestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root, in particular, are preferred.

Particular preference is given to the extracts from green tea, oak bark, stinging nettle, hamamelis, hops, chamomile, burdock, horsetail, linden blossom, almond, aloe vera, coconut, mango, apricot, lemon, wheat, kiwi, melon, orange, grapefruit, sage, rosemary, birch, lady's smock, wild thyme, yarrow, restharrow, meristem, ginseng and ginger root.

Of very particular suitability for the use according to the invention are the extracts from green tea, almond, aloe vera, coconut, mango, apricot, lemon, wheat, kiwi and melon.

Extractants which can be used for producing the specified plant extracts are water, alcohols, and mixtures thereof. Among the alcohols, lower alcohols such as ethanol and isopropanol, but in particular polyhydric alcohols, such as ethylene glycol and propylene glycol, either as the sole extractant or in a mixture with water, are preferred here. Plant extracts based on water/propylene glycol in the ratio 1:10 to 10:1 have proven to be particularly suitable.

According to the invention, the plant extracts can be used either in pure form or in dilute form. If they are used in dilute form, they usually comprise about 2-80% by weight of active substance and, as solvent, the extractant or extractant mixture used in their recovery.

In addition, it may be preferred to use mixtures of two or more, in particular of two, different plant extracts in the preparations according to the invention.

Furthermore, it is preferred according to the invention to use hydroxycarboxylic acids and here in turn in particular the dihydroxy-, trihydroxy- and polyhydroxycarboxylic acids, and also the dihydroxy-, trihydroxy- and polyhydroxy-di-, tri- and polycarboxylic acids. In this connection it has been found that, besides the hydroxycarboxylic acids, the hydroxycarboxylic acid esters, and the mixtures of hydroxycarboxylic acids and esters thereof and also polymeric carboxylic acids and esters thereof may be very particularly preferred. Preferred hydroxycarboxylic acid esters are, for example, full esters of glycolic acid, lactic acid, malic acid, tartaric acid or citric acid. Further hydroxycarboxylic acid esters which are suitable in principle are esters of β-hydroxypropionic acid, or tartronic acid, of D-gluconic acid, of sugar acid, of mucic acid or of glucuronic acid. Suitable alcohol components of these esters are primary, linear or branched aliphatic alcohols having 8-22 carbon atoms, e.g. fatty alcohols or synthetic fatty alcohols. Here, the esters of C12-C15 fatty alcohols are particularly preferred. Esters of this type are commercially available, e.g. under the trade name Cosmacol® from EniChem, Augusta Industriale. Particularly preferred polyhydroxypolycarboxylic acids are polylactic acid and polytartaric acid, and esters thereof.

In a further preferred embodiment, emulsifiers are used in the preparations of the method according to the invention. At the phase interface, emulsifiers bring about the formation of water-stable or oil-stable adsorption layers which protect the dispersed droplets against coalescence and thus stabilize the emulsion. Emulsifiers are therefore constructed like surfactants from one hydrophobic molecular moiety and one hydrophilic molecular moiety. Hydrophilic emulsifiers form preferably O/W emulsions and hydrophobic emulsifiers form preferably W/O emulsions. An emulsion is understood as meaning a droplet-like distribution (dispersion) of one liquid in another liquid with expenditure of energy to create stabilizing phase interfaces by means of surfactants. The selection of these emulsifying surfactants or emulsifiers is governed by the substances to be dispersed and the particular external phase, and also the finely divided nature of the emulsion. Extensive definitions and properties of emulsifiers are given in “H.-D. Dorfler, Grenzflachen- und Kolloidchemie [Interface and colloid chemistry], VCH Verlagsgesellschaft mbH. Weinheim, 1994”. Emulsifiers which can be used according to the invention are, for example,
addition products of from 4 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂-fatty acid monoesters and diesters of addition products of from 1 to 30 mol of ethylene oxide onto polyols having 3 to 6 carbon atoms, in particular onto glycerol,
ethylene oxide and polyglycerol addition products onto methyl glucoside fatty acid esters, fatty acid alkanolamides and fatty acid glucamides,
C₈-C₂₂-alkyl mono- and oligoglycosides and ethoxylated analogs thereof, preference being given to degrees of oligomerization of from 1.1 to 5, in particular 1.2 to 2.0, and glucose as sugar component,
mixtures of alkyl (oligo)glucosides and fatty alcohols, for example the commercially available product Montanov® 68,
addition products of from 5 to 60 mol of ethylene oxide onto castor oil and hydrogenated castor oil,
partial esters of polyols having 3-6 carbon atoms with saturated fatty acids having 8 to 22 carbon atoms,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols can also be isolated from fungi and yeasts, the so-called mycosterols.
phospholipids. These are understood primarily as meaning the glucose phospholipids which are obtained, for example, as lecithins and phosphatidylcholines from e.g. egg yolk or plant seeds (e.g. soybeans).
fatty acid esters of sugars and sugar alcohols, such as sorbitol,
polyglycerols and polyglycerol derivatives, such as, for example, polyglycerol poly-12-hydroxystearate (commercial product Dehymuls® PGPH),
linear and branched fatty acids having 8 to 30 carbon atoms and the Na, K, ammonium, Ca, Mg and Zn salts thereof.

The preparations according to the invention comprise the emulsifiers preferably in amounts of 0.1-25% by weight, in particular 0.1-3% by weight, based on the total preparation.

Preferably, the preparations according to the invention can comprise at least one nonionogenic emulsifier with an HLB value of from 8 to 18, according to the definitions given in Römpp Lexikon Chemie [Römpp Chemistry Lexikon] (Ed. J. Falbe, M. Regitz), 10th edition, Georg Thieme Verlag Stuttgart, New York, (1997), page 1764. Nonionogenic emulsifiers with an HLB value of 10-15 may be particularly preferred according to the invention.

Further active substances which may be used are heterocyclic compounds such as, for example, derivatives of imidazole, pyrrolidine, piperidine, dioxolane, dioxane, morpholine and piperazine. Also suitable are derivatives of these compounds, such as, for example, the C₁₋₄-alkyl derivatives, C₁₋₄-hydroxyalkyl derivatives and C₁₋₄-aminoalkyl derivatives. Preferred substituents which may be positioned either on carbon atoms or on nitrogen atoms of the heterocyclic ring systems are methyl, ethyl, β-hydroxyethyl and β-aminoethyl groups. Preferably, these derivatives contain 1 or 2 of these substituents.

Derivatives of heterocyclic compounds preferred according to the invention are, for example, 1-methylimidazole, 2-methylimidazole, 4(5)-methylimidazole, 1,2-dimethylimidazole, 2-ethylimidazole, 2-isopropylimidazole, N-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine. Furthermore, imidazole derivatives preferred according to the invention are biotin, hydantoin and benzimidazole.

Among these heterocyclic active substances, the mono- and dialkylimidazoles, biotin and hydantoin are particularly preferred.

These heterocyclic compounds are present in the preparations according to the invention in amounts of from 0.5 to 10% by weight, based on the total preparation. Amounts of from 2 to 6% by weight have proven to be particularly suitable.

According to the invention, further active substances which may be present in all of the aqueous preparations used according to the invention are amino acids and amino acid derivatives. From the group of amino acids, arginine, citrulin, histidine, ornithine and lysine in particular have proven to be suitable according to the invention. The amino acids can be used either as free amino acid, or as salts, e.g. as hydrochlorides. Furthermore, oligopeptides of on average 2-3 amino acids which have a high fraction (>50%, in particular >70%) of the specified amino acids have also proven useful according to the invention.

According to the invention, particular preference is given to arginine and its salts and arginine-rich oligopeptides.

These amino acids and derivatives are present in the preparations according to the invention in amounts of from 0.5 to 10% by weight, based on the total preparation. Amounts of from 2 to 6% by weight have proven to be particularly suitable.

Additionally, it can prove advantageous if penetration auxiliaries and/or swelling agents are present in the preparations according to the invention. To be included here are, for example, urea and urea derivatives, guanidine and derivatives thereof, arginine and derivatives thereof, waterglass, imidazole and derivatives thereof, histidine and derivatives thereof, benzyl alcohol, glycerol, glycol and glycol ether, propylene glycol and propylene glycol ether, for example propylene glycol monoethyl ether, carbonates, hydrogen carbonates, diols and triols, and in particular 1,2-diols and 1,3-diols, such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol. The penetration auxiliaries and swelling agents are present in the preparations used according to the invention in amounts of from 0.1 to 20% by weight, based on the total preparation. Amounts of from 01 to 10% by weight are preferred.

Furthermore, the preparations according to the invention can, particularly if they are waving lotions, comprise components which enhance the waving strength, in particular urea, imidazole and the abovementioned diols. For further information regarding such components which enhance waving strength, reference is made to the publications DE-A 44 36 065 and EP-B1-363 057, to the contents of which reference is expressly made.

The compounds which enhance waving strength may be present in the preparations according to the invention in amounts of from 0.5 to 5% by weight, based on the total preparation. Amounts of from 1 to 4% by weight have proven sufficient, for which reason these amounts are particularly preferred.

In step (b) of the method according to the invention, besides the at least one oxidizing agent, such as, for example, hydrogen peroxide, use is preferably also made of a stabilizer customary for stabilizing aqueous hydrogen peroxide preparations. The pH of such aqueous H₂O₂ preparations, which usually comprise about 0.5 to 15% by weight, ready-to-use generally about 0.5-3% by weight, of H₂O₂, is preferably 2 to 6, in particular 2 to 4; it is adjusted using acids, preferably phosphoric acid, phosphonic acids and/or dipicolinic acid. Bromate-based neutralizers comprise the bromates usually in concentrations of from 1 to 10% by weight and the pH of the solutions is adjusted to 4 to 7. According to the invention, particular preference may be given to the use of neutralizer concentrates, which are diluted with water prior to use. Neutralizers for the permanent shaping of keratin-containing fibers are often formulated as solids. They then comprise the oxidizing agent in the form of a solid, e.g. sodium perborate. Shortly prior to use, these agents are then admixed with water to form the aqueous preparations.

Furthermore, it is possible to carry out the oxidation with the help of enzymes, where the enzymes are used both for producing oxidizing percompounds, and also for enhancing the effect of small amounts of oxidizing agent present, or else enzymes are used which transfer electrons from suitable developer components (reducing agents) to atmospheric oxygen. Preference is given here to oxidases, such as tyrosinase, ascorbate oxidase and laccase, but also glucose oxidase, uricase or pyruvate oxidase. In addition, mention may be made of the procedure to enhance the effect of small amounts (e.g. 1% and below, based on the total composition) of hydrogen peroxide through peroxidases.

The neutralizers according to the invention can also be formulated as solids. They then comprise the oxidizing agent in the form of a solid, e.g. potassium or sodium bromate. It is likewise possible and preferred to formulate the oxidizing agent as a 2-component system. The two components, of which one is preferably a hydrogen peroxide solution or an aqueous solution of another oxidizing agent, and the other comprises the customary constituents, in particular care substances and/or reducing agents, are likewise only mixed shortly prior to use.

Furthermore, silicones are suitable as conditioning active substances. Silicones which can be used according to the invention are preferably linear, cyclic or branched silicones chosen from the cyclomethicone, dimethiconol, dimethicone copolyols, amodimethicone, trimethylsilylamodimethicone and phenyltrimethicone types. These types of silicone are known to the person skilled in the art under the nomenclature of the Cosmetic, Toiletry and Fragrance Association (CTFA) and disclosed in: M. D. Berthiaume, Society of the Cosmetic Chemists Monograph Series, “Silicones in Hair Care”, Ed.: L. D. Rhein, published: Society of the Cosmetic Chemists, 1997, Chapter 2, to which reference is explicitly made at this point. Polysiloxanes, such as dialkyl- and alkylarylsiloxanes, for example dimethylpolysiloxane and methylphenylpolysiloxane, and alkoxylated analogs thereof, analogs terminated with hydroxyl groups and quaternized analogs, and cyclic siloxanes. Here, particularly the silicones with the INCI names Dimethicone, PEG-12 Dimethicone, PEG/PPG-18/18 Dimethicone, Cyclomethicone, Dimethiconol, Quarternium-80 and Amodimethicone, and mixtures thereof are particularly preferred silicones.

Examples of such silicones are the products sold by Dow Corning under the names DC 190 (INCI name: PEG/PPG-18/18 Dimethicone), DC 193 (INCI name: PEG-12 Dimethicone), DC 200, DC 1401 (INCI name: Cyclomethicone, Dimethiconol) and DC 1403 (INCI name: Dimethicone, Dimethiconol), and the commercial products DC 244 (INCI name: Cyclomethicone), DC 344 (INCI name: Cyclomethicone) and DC 345 (INCI name: Cyclomethicone) from Dow Corning, Q2-7224 (manufacturer: Dow Corning; a stabilized trimethylsilylamodimethicone, Dow Corning 929 emulsion (comprising a hydroxylamino-modified silicone, which is also referred to as amodimethicone), SM-2059 (manufacturer: General Electric), SLM-55067 (manufacturer: Wacker), Abil Quat 3270 and 3272 (manufacturer: Th. Goldschmidt; diquaternary polydimethylsiloxanes, INCI name: Quaternium-80), and the commercial product Fancorsil® LIM-1. A suitable anionic silicone oil is the product Dow Corning® 1784.

Further active ingredients, auxiliaries and additives are, for example,
thickeners, such as agar-agar, guar gum, alginates, xanthan gum, gum Arabic, karaya gum, carob seed flour, linseed gums, dextrans, cellulose derivatives, e.g. methylcellulose, hydroxyalkylcellulose and carboxymethylcellulose, starch fractions and derivatives such as amylose, amylopectin and dextrins, clays, such as, for example, bentonite or completely synthetic hydrocolloids such as, for example, polyvinyl alcohol,
hair-conditioning compounds, such as phospholipids, for example, soya lecithin, egg lecithin and cephalins, and silicone oils,
perfume oils, dimethyl isosorbide and cyclodextrins,
solvents and solubility promoters, such as ethanol, isopropanol, ethylene glycol, propylene glycol, glycerol and diethylene glycol,
active ingredients which improve fiber structure, in particular mono-, di- and oligosaccharides, such as, for example, glucose, galactose, fructose, fruit sugar and lactose,
conditioning active ingredients, such as paraffin oils, vegetable oils, e.g. sunflower oil, orange oil, almond oil, wheatgerm oil and peach kernel oil, and
quaternized amines, such as methyl-1-alkylamidoethyl-2-alkylimidazolinium methosulfate,
antifoams, such as silicones,
dyes for coloring the composition,
antidandruff active ingredients, such as piroctone olamine, zinc omadine and climbazole,
active ingredients such as allantoin and bisabolol,
cholesterol,
consistency regulators, such as sugar esters, polyol esters or polyol alkyl ethers,
fats and waxes, such as spermaceti, beeswax, montan wax and paraffins,
fatty acid alkanolamides,
swelling and penetration substances, such as primary, secondary and tertiary phosphates,
opacifiers, such as latex, styrene/PVP and styrene/acrylamide copolymers
pearlizing agents, such as ethylene glycol mono- and distearate, and PEG-3 distearate,
pigments,
propellants, such as propane/butane mixtures, N₂O, dimethyl ether, CO₂ and air,
antioxidants.

With regard to further optional components and the amounts of these components used, reference is expressly made to the relevant handbooks known to the person skilled in the art, e.g. the abovementioned monograph by K. H. Schrader.

The preparations according to the invention can be used in compositions for haircare, such as shampoos, conditioners, rinses, aerosols and gels, or else are used in compositions for the treatment of textiles or fibers in the form of detergents, softeners, impregnations and finishes.

Application of the polysulfide (I) and of the reducing agent to the fiber to be treated can take place successively in any order or after prior mixing of polysulfide (I) and reducing agent.

In one embodiment of the invention, the polysulfide (I) and the reducing agent, and optionally also the oxidizing agent and optionally further constituents of the preparations being used are provided separately from one another in a kit-of-parts. The individual components may be present in mixed, dissolved, dispersed or emulsified form in a suitable carrier. Preferably, the kit-of-parts consists of a preparation comprising the polysulfide (I) and/or at least one preparation as has been described in the preceding text as preparations V, Z, R O, where at least one of the preparations present in the kit-of-parts comprises a polysulfide of the formula (I).

The invention thus further provides a kit for use in a method according to the invention, comprising at least
(a) one of the preparations described above, comprising at least one polysulfide of the formula (I), and
(b) a preparation comprising a reducing agent, in particular a keratin-reducing compound,
   where the preparations are packaged separately.

The invention likewise provides a kit for use in a method according to the invention, additionally including a preparation comprising an oxidizing agent.

Furthermore, the present invention relates to a fiber, in particular a keratin fiber, which is obtainable by the method described above.

The invention further provides the use of a polysulfide of the formula (I) for restructuring keratin fibers, in particular hair, where restructuring involves, in particular, fiber strengthening.

The examples below are intended to illustrate the invention in more detail.

### EXAMPLES

Unless stated otherwise, all of the percentages are % by weight, and all of the amounts are parts by weight.

### Synthesis Examples

### Synthesis Example 1

### Preparation of a Polysulfide of the Formula (I) From Cystine and Succinyl Dichloride by Interfacial Condensation

0.5 mol of succinyl dichloride was dissolved in 1 l of dichloromethane. 0.5 mol of cystine was dissolved in 1 l of 2N NaOH and cooled with ice water. The two solutions were combined and dispersed with vigorous stirring. After a short time, a solid started to precipitate out at the interface. After about 1 h, the succinyl dichloride had fully reacted and the reaction was complete. The solid was then filtered off and washed with ice-cold water and with dichloromethane. Yield: 86 g of a beige-yellowish solid.

### Synthesis Example 2

### Preparation of a Polysulfide of the Formula (I) From Cystine and Succinyl Dichloride by Interfacial Condensation

Succinyl dichloride (0.07 mol) was dissolved in 50 ml of dichloromethane. Cystine (0.05 mol) was dissolved in 100 ml of 1N NaOH, initially introduced into the reaction vessel and cooled with ice water. The two solutions were combined and dispersed with vigorous stirring. After a short time, a solid started to precipitate out at the interface. As soon as the acid chloride had fully reacted, the reaction was terminated. The precipitated solid was filtered off, washed with ice-cold water and with dichloromethane and dried under reduced pressure. Yield 8.2 g of a slightly yellowish powder. The resulting product contained cystine.

### Synthesis Example 3

### Preparation of a Polysulfide of the Formula (I) From Cystine and Succinyl Dichloride by Interfacial Condensation

Succinyl dichloride (0.05 mol) was dissolved in 50 ml of dichloromethane. Cystine (0.05 mol) was dissolved in 100 ml of 2N NaOH, initially introduced into the reaction vessel and cooled with ice water. The two solutions were combined and dispersed with vigorous stirring. The formation of a solid could be observed at the interface. When the reaction was complete, the solid was isolated and the organic phase was separated from the aqueous phase. The aqueous phase was then combined with the solid and freeze-dried. Yield: 15.3 g of beige-yellow powder. The resulting product contained cystine and succinic acid.

### Synthesis Example 4

### Preparation of a Polysulfide of the Formula (I) From Cystine Dimethyl Ester and Succinyl Dichloride

The synthesis was carried out as in U.S. Pat. No. 5,646,239, with firstly the polysulfide polymethyl ester “stage I” being obtained (corresponding to U.S. Pat. No. 5,646,239, Example 2), which was then saponified to the polysulfide “stage II” (corresponding to U.S. Pat. No. 5,646,239, Example 3).

### Application Examples

Elongation at Break Experiments on Hair

To show the effects according to the invention, the substances obtained according to the synthesis examples were investigated within the framework of a cold-waving process (hair type: natural dark brown code #6634 from Alkinco). Here, the contact times in the permanent waving process remained unchanged. The treatment temperature was 32° C.

1. Measurement Apparatuses

To demonstrate the effects according to the invention, stress values, gradients, modulus of elasticity, elongation at break and yield stress of the wet hair were determined using a stress-strain device from Dia-Stron (MTT 670). The hair cross section area of the individual wet hairs was determined by means of contactless projection measurement by laser technology known in the prior art. For this, a universal dimensiometer model UMD5000A from Zimmer was used.

2. Statistical Evaluation

The t-test, a statistical evaluation with which the measurement series are bilaterally compared pairwise, gives percentage probabilities of the measurement series being different (difference 90-95%: measurement series have a tendency to be different, >95%: measurement series differ significantly, >99%: highly significant differences in the measurement series).

3. Restructuring by Polysulfides

### 3.1 Hair Treatment

40 individual hairs were divided into two parts. One part was damaged by two cold waves, the other part was treated with two cold waves in whose preparation the reducing agent was admixed with the product from synthesis example 1 about 2 minutes prior to application to the hair. All 80 individual hairs were subjected to a hair cross section area determination in the wet state prior to determining the breaking curves.

### 3.2 Treatment Steps:

In the experiments below, the following treatment steps were used.
a) 30 min application of a cold wave (7% TGA=thioglycolic acid, 0.3% Turpinal SL=1-hydroxyethane-1,1-diphosphonic acid, 3.5% (NH₄)₂CO₃, pH 8.4).
The hairs were then rinsed for 5 minutes with water.
b) 10 min application of the neutralizer (2% H₂O₂, 1% Turpinal SL, pH 4.0). The hairs were then rinsed for 5 minutes with water.
c) Storage of the hair for at least 24 h at RT (about 20° C.)
d) Measurement of the hair cross sections of the individual wet hairs.
e) Determination of the breaking values of the individual wet hairs.
f) 30 minute application of a cold wave (12% ammonium thioglycolate, 5% NH₄HCO₃, 0.5% NH₃, 1% Cremophor RH 40, 1% Lamepon S, 0.5% perfume).
The hairs are then rinsed with water for 5 minutes.
g) 10 minute application of a neutralizer diluted 1:1 with water prior to use (5% H₂O₂, 0.2% NH₃, 1.7% Turpinal SL, 6% Texapon NSO UP (sodium lauryl ether sulfate), water completely demineralized ad 100%).
The hairs are then rinsed with water for 5 minutes.

### 3.3 Results of Damage Potential of a Cold Wave (Comparison):

Reference example: untreated, i.e. healthy and undamaged hair.

Comparison example: 2× permanently waved, i.e. damaged hair, as described under 3.2: steps a), b), c); repetition of steps a), b), c); then d) and e).

The influence of the permanent wave on hair was investigated by means of stress-strain measurement in the wet state.

| | | | | | |
|---|---|---|---|---|---|
| Hair cross | | | Stress | Stress at | Work at |
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |

Untreated

| | | | | | |
|---|---|---|---|---|---|
| 4.13E+03 | 1.64E+09 | 2.22E−01 | 5.93E−05 | 5.62E−05 | 8.79E−04 |

2x permanently waved

| | | | | | |
|---|---|---|---|---|---|
| 4.70E+03 | 7.48E+08 | 1.13E−01 | 2.71E−05 | 2.49E−05 | 4.42E−04 |

t-Test, bilateral, pairwise

| | | | | | |
|---|---|---|---|---|---|
| Signifi- | Significantly | Signifi- | Signifi- | Signifi- | Signifi- |
| cantly | different | cantly | cantly | cantly | cantly |
| different | | different | different | different | different |

| | | | | |
|---|---|---|---|---|
| Stress at | Work at | | | |
| 25% | 25% | Elongation | | |
| elongation | elongation | at break | Yield stress | Total work |
| [N/μm²] | [J] | [%] | [N/μm²] | [J] |

Untreated

| | | | | |
|---|---|---|---|---|
| 7.14E−05 | 1.67E−03 | 5.48E+01 | 1.97E−04 | 6.92E−03 |

2x permanently waved

| | | | | |
|---|---|---|---|---|
| 3.22E−05 | 8.52E−04 | 6.05E+01 | 1.27E−04 | 4.77E−03 |

t-Test, bilateral, pairwise

| | | | | |
|---|---|---|---|---|
| Significantly | Significantly | Significantly | Significantly | Significantly |
| different | different | different | different | different |

Damage to the hair by the permanent waving method is clearly evident from the increase in the hair cross section area, the reduction in the modulus of elasticity, in the gradient, in the stress values, and also in the work values. In addition, an increase in the elongation at break is observed.

Under the influence of substances with a restructuring effect, the parameter changes which characterize hair damage should be reduced, i.e. there should be a change in the direction of the parameter values typical of untreated and undamaged hair. This was confirmed in the following investigations in which the method according to the invention was used.

3.4 Results for Cold Wave with 2% of the Product from Synthesis Example 1
Reference example: 2× permanently waved: as described under 3.2 steps a), b), c); repetition of steps a), b), c); then d) and e).

Example according to the invention: 2× permanently waved hair with 2% of the product from synthesis example 1 in the cold wave, as described under 3.2: steps a) with 2% product from synthesis example 1, b), c); repetition of steps a) with 2% product from synthesis example 1, b), c); then d) and e).

The effect of the composition according to the invention on hair was investigated by means of stress-strain measurement in the wet state.

| | | | | | |
|---|---|---|---|---|---|
| Hair cross | | | Stress | Stress at | Work at |
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |

Reference damaged 2x by CW

| | | | | | |
|---|---|---|---|---|---|
| 5.20E+03 | 7.42E+08 | 1.24E−01 | 2.57E−05 | 2.37E−05 | 4.56E−04 |

Example according to the invention 2x cold-waved
with addition of 2% product from synthesis example 1

| | | | | | |
|---|---|---|---|---|---|
| 4.83E+03 | 1.08E+09 | 1.66E−01 | 3.50E−05 | 3.36E−05 | 6.25E−04 |

t-Test, pairwise, bilateral

| | | | | | |
|---|---|---|---|---|---|
| Signifi- | Significantly | Signifi- | Signifi- | Signifi- | Signifi- |
| cantly | different | cantly | cantly | cantly | cantly |
| different | | different | different | different | different |

| | | | | |
|---|---|---|---|---|
| Stress at | Work at | | | |
| 25% | 25% | Elongation | | |
| elongation | elongation | at break | Yield stress | Total work |
| [N/μm²] | [J] | [%] | [N/μm²] | [J] |

Reference damaged 2x by CW

| | | | | |
|---|---|---|---|---|
| 2.92E−05 | 8.65E−04 | 6.14E+01 | 1.19E−04 | 4.82E−03 |

Example according to the invention 2x cold-waved with
addition of 2% product from synthesis example 1

| | | | | |
|---|---|---|---|---|
| 4.10E−05 | 1.17E−03 | 5.45E+01 | 1.36E−04 | 5.02E−03 |

t-Test, pairwise, bilateral

| | | | | |
|---|---|---|---|---|
| Significantly | Significantly | Significantly | Significantly | Not |
| different | different | different | different | different |

Result: by adding the product from the synthesis example 1, it was possible to establish a significant improvement in hair structure compared to the reference. A significant increase in the modulus of elasticity, in the gradient, in the stress values, in the work values, and a reduction in elongation at break, and in the hair cross section area was observed.

3.5 Results for Cold Wave with 1% of the Product from Synthesis Example 1

Reference example: 2× permanently waved: as described under 3.2 steps a), b), c); repetition of steps a), b), c); then d) and e).

Example according to the invention: 2× permanently waved hair with 1% product from synthesis example 1 in the cold wave, as described under 3.2: steps a) with 1% product from synthesis example 1, b), c); repetition of steps a) with 1% product from synthesis example 1, b), c); then d) and e).

The influence of the composition according to the invention on hair was investigated by means of stress-strain measurement in the wet state.

| | | | | |
|---|---|---|---|---|
| | | Stress | Stress at | Work at |
| Modulus of | Elastic | plateau | 15% | 15% |
| elasticity | gradient | range | elongation | elongation |
| [N/m²] | [N/mm] | [N/μm²] | [N/μm²] | [J] |

Reference 2x damaged by CW

| | | | | |
|---|---|---|---|---|
| 7.82E+08 | 1.17E−01 | 2.69E−05 | 2.54E−05 | 4.44E−04 |

Example according to the invention 2x cold-waved with addition of
1% product from synthesis example 1

| | | | | |
|---|---|---|---|---|
| 9.26E+08 | 1.36E−01 | 3.05E−05 | 2.97E−05 | 5.30E−04 |

t-Test, pairwise, bilateral

| | | | | |
|---|---|---|---|---|
| Significantly | Significantly | Significantly | Significantly | Significantly |
| different | different | different | different | different |

| | | |
|---|---|---|
| Stress at | Work at | |
| 25% | 25% | Elongation |
| elongation | elongation | at break |
| [N/μm²] | [J] | [%] |

Reference 2x damaged by CW

| | | |
|---|---|---|
| 3.11E−05 | 8.37E−04 | 5.86E+01 |

Example according to the invention 2x cold-waved with addition
of 1% product from synthesis example 1

| | | |
|---|---|---|
| 3.64E−05 | 9.99E−04 | 5.33E+01 |

t-Test, pairwise, bilateral

| | | |
|---|---|---|
| Significantly | Significantly | Significantly |
| different | different | different |

Result: by adding the product from synthesis example 1, a significant improvement in the hair structure compared to the reference was established. A significant increase in the modulus of elasticity, in the gradient, in the stress values, in the work values, and a reduction in the elongation at break was observed.

The results under 3.4 and 3.5 show that in all parameters, such as gradient, modulus of elasticity, stress values, elongation at break and work, by applying the product from synthesis example 1, up to 75% of the values for untreated hair, depending on the concentration, are achieved. Compared to damaged hair, an improvement by up to 50% is achieved through the treatment.

3.6 Results for Cold Wave with 1% Cystine
Reference example: 2× permanently waved: as described under 3.2 steps a), b), c); repetition of steps a), b), c); then d) and e).

Comparative example with cystine: 2× permanently waved hair, as described under 3.2: steps a) with 1% cystine, b), c); repetition of steps a) with 1% cystine, b), c); then d) and e).

| | | | | | |
|---|---|---|---|---|---|
| Hair cross | | | Stress | Stress at | Work at |
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |

Reference 2x damaged by CW

| | | | | | |
|---|---|---|---|---|---|
| 4.76E+03 | 7.07E+08 | 1.08E−01 | 2.46E−05 | 2.33E−05 | 4.26E−04 |

Comparative example 2x cold-waved with addition of 1% cystine

| | | | | | |
|---|---|---|---|---|---|
| 4.97E+03 | 7.81E+08 | 1.22E−01 | 2.62E−05 | 2.54E−05 | 4.80E−04 |

t-Test, pairwise, bilateral

| | | | | | |
|---|---|---|---|---|---|
| Not | Significantly | Signifi- | Signifi- | Signifi- | Signifi- |
| different | different | cantly | cantly | cantly | cantly |
| | | different | different | different | different |

| | | | | |
|---|---|---|---|---|
| Stress at | Work at | | | |
| 25% | 25% | Elongation | | |
| elongation | elongation | at break | Yield stress | Total work |
| [N/μm²] | [J] | [%] | [N/μm²] | [J] |

Reference 2x damaged by CW

| | | | | |
|---|---|---|---|---|
| 2.88E−05 | 8.08E−04 | 5.87E+01 | 1.15E−04 | 4.12E−03 |

Comparative example 2x cold-waved with addition of 1% cystine

| | | | | |
|---|---|---|---|---|
| 3.15E−05 | 9.10E−04 | 5.45E+01 | 1.09E−04 | 3.95E−03 |

t-Test, pairwise, bilateral

| | | | | |
|---|---|---|---|---|
| Significantly | Significantly | Significantly | Not | Not |
| different | different | different | different | different |

Statistical evaluation: to demonstrate the effect of the product from synthesis example 1 compared to the effect of L-cystine, the two treatment groups were compared taking into consideration their starting values (only cold-waved) with the covariance analysis based on multiple comparisons in accordance with Scheffèe.

The following significances were established:

| Hair cross | | | Stress | Stress at | Work at |
|---|---|---|---|---|---|
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |
| Not | Significantly | Not | Signifi- | Signifi- | Signifi- |
| different | different | different | cantly | cantly | cantly |
| | | | different | different | different |

| Stress at | Work at | | | |
|---|---|---|---|---|
| 25% | 25% | Elongation | | |
| elongation | elongation | at break | Yield stress | Total work |
| [N/μm²] | [J] | [%] | [N/μm²] | [J] |
| Significantly | Significantly | Not | Not | Not |
| different | different | different | different | different |

Result: by means of covariance analysis, a significantly greater restructuring of the hair through the product from synthesis example 1 compared to L-cystine was demonstrated.

3.7 Results in a Standard Commercial Cold Wave with 1% Product From Synthesis Example 1:
Reference example: 2× permanently waved with a standard commercial permanent wave: as described under 3.2 steps f), g), c); repetition of steps f), g), c); then d) and e).

Example according to the invention: 2× permanently waved hair with 1% product from synthesis example 1 in the cold wave, as described under 3.2: steps f) with 1% product from synthesis example 1, g), c); repetition of steps f) with 1% product from synthesis example 1, g), c); then d) and e).

The influence of the composition according to the invention on hair was investigated by means of stress-strain measurement in the wet state.

| | | | | |
|---|---|---|---|---|
| | | Stress | Stress at | Work at |
| Modulus of | Elastic | plateau | 15% | 15% |
| elasticity | gradient | range | elongation | elongation |
| [N/m²] | [N/mm] | [N/μm²] | [N/μm²] | [J] |

Reference 2x damaged by CW

| | | | | |
|---|---|---|---|---|
| 8.94E+08 | 1.28E−01 | 3.09E−05 | 2.96E−05 | 5.00E−04 |

Comparative example 2x cold-waved with addition of 1% product
from synthesis example 1

| | | | | |
|---|---|---|---|---|
| 1.01E+09 | 1.43E−01 | 3.43E−05 | 3.25E−05 | 5.48E−04 |

t-Test, pairwise, bilateral

| | | | | |
|---|---|---|---|---|
| Significantly | Significantly | Significantly | Significantly | Significantly |
| different | different | different | different | different |

| | | | |
|---|---|---|---|
| Stress at | Work at | | |
| 25% | 25% | | |
| elongation | elongation | Yield stress | Total work |
| [N/μm²] | [J] | [N/μm²] | [J] |

Reference 2x damaged by CW

| | | | |
|---|---|---|---|
| 3.83E−05 | 9.61E−04 | 1.33E−04 | 4.28E−03 |

Comparative example 2x cold-waved with addition of
1% product from synthesis example 1

| | | | |
|---|---|---|---|
| 4.10E−05 | 1.04E−03 | 1.41E−04 | 4.78E−03 |

t-Test, pairwise, bilateral

| | | | |
|---|---|---|---|
| Significantly | Significantly | Significantly | Significantly |
| different | different | different | different |

Result: by adding the product from synthesis example 1, a significant improvement in the hair structure in comparison to the reference was established. A significant increase in the modulus of elasticity, in the gradient, in the stress values, in the work values was observed.

3.8 Results in a Standard Commercial Cold Wave with 1% Succinic Acid:

Reference example: 2× permanently waved with a standard commercial permanent wave: as described under 3.2 steps f), g), c); repetition of steps f), g), c); then d) and e).

Example according to the invention: 2× permanently waved hair with 1% succinic acid in the cold wave, as described under 3.2: steps f) with 1% succinic acid, g), c); repetition of steps f) with 1% succinic acid, g), c); then d) and e).

| | | | | | |
|---|---|---|---|---|---|
| Hair cross | | | Stress | Stress at | Work at |
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |

Reference 2x damaged by CW

| | | | | | |
|---|---|---|---|---|---|
| 4.51E+03 | 8.09E+08 | 1.18E−01 | 2.83E−05 | 2.72E−05 | 4.65E−04 |

Comparative example 2x cold-waved with addition of 1% succinic acid

| | | | | | |
|---|---|---|---|---|---|
| 4.49E+03 | 8.76E+08 | 1.26E−01 | 3.08E−05 | 2.91E−05 | 5.02E−04 |

t-Test, bilateral, pairwise

| | | | | | |
|---|---|---|---|---|---|
| Not | Significantly | Signifi- | Signifi- | Signifi- | Signifi- |
| different | different | cantly | cantly | cantly | cantly |
| | | different | different | different | different |

| | | | | |
|---|---|---|---|---|
| Stress at | Work at | | | |
| 25% | 25% | Elongation | | |
| elongation | elongation | at break | Yield stress | Total work |
| [N/μm²] | [J] | [%] | [N/μm²] | [J] |

Reference 2x damaged by CW

| | | | | |
|---|---|---|---|---|
| 3.49E−05 | 8.94E−04 | 5.52E+01 | 1.22E−04 | 4.14E−03 |

Comparative example 2x cold-waved with addition of 1% succinic acid

| | | | | |
|---|---|---|---|---|
| 3.69E−05 | 9.58E−04 | 5.69E+01 | 1.34E−04 | 4.60E−03 |

t-Test, bilateral, pairwise

| | | | | |
|---|---|---|---|---|
| Significantly | Significantly | Not | Significantly | Significantly |
| different | different | different | different | different |

Statistical evaluation: to assess the effect of succinic acid compared to the effect of the product from synthesis example 1, the two treatment groups were compared taking into consideration their starting values (only cold-waved) with the covariance analysis based on multiple comparisons in accordance with Scheffèe.

The following significances were established:

| Hair cross | | | Stress | Stress at | Stress at |
|---|---|---|---|---|---|
| section | Modulus of | Elastic | plateau | 15% | 25% |
| area | elasticity | gradient | range | elongation | elongation |
| Not | Significantly | Signifi- | Signifi- | Signifi- | Signifi- |
| different | different | cantly | cantly | cantly | |
| | | different | different | different | different |

Result: succinic acid on its own likewise leads to a significant strengthening of hair. Compared to the product from synthesis example 1, however, a clear advantage is evident for the product from synthesis example 1 since in the case of the modulus of elasticity and the stress values of the plastic range, the measurement series are significantly different.

3.9 Results in a Standard Commercial Cold Wave with 1% Product From Synthesis Example 3:

Reference example: 2× permanently waved with a standard commercial permanent wave: as described under 3.2 steps f)¹⁾, g), c); repetition of steps f)¹⁾, g), c); then d) and e).

Comparative example: 2× permanently waved hair with 1% succinic acid in the cold wave, as described under 3.2: steps f)¹⁾, with 1% product from synthesis example 3, g), c); repetition of steps f)¹⁾ with 1% product from: synthesis example 3, g), c); then d) and e).

¹⁾In example 3.9, the cold wave formulation described under 3.2 f) was replaced by the following cold wave formulation: 17% ammonium thioglycolate (71% strength), 0.3% Turpinal SL, 2.5% ammonia (25% strength), 5% ammonium hydrogen carbonate, 1% Cremophor RH 40, 1% Lamepon S, 0.5% perfume, 0.1% Gluadin WQ (laurdimonium hydroxypropyl hydrolyzed wheat protein), 0.1% Merquat 100 (polydimethyldiallylammonium chloride), water ad 100.

The influence of the product according to the invention on hair was investigated by means of stress-strain measurement in the wet state.

| | | | | |
|---|---|---|---|---|
| | | Stress | Stress at | Work at |
| Modulus of | Elastic | plateau | 15% | 15% |
| elasticity | gradient | range | elongation | elongation |
| [N/m²] | [N/mm] | [N/μm²] | [N/μm²] | [J] |

Reference 2x damaged by cold wave

| | | | | |
|---|---|---|---|---|
| 7.09E+08 | 1.02E−01 | 2.66E−05 | 2.56E−05 | 4.10E−04 |

Example according to the invention 2x with cold wave with the
addition of 1% product from synthesis example 3

| | | | | |
|---|---|---|---|---|
| 8.43E+08 | 1.21E−01 | 3.00E−05 | 2.86E−05 | 4.62E−04 |

t-Test, bilateral, pairwise

| | | | | |
|---|---|---|---|---|
| Significantly | Significantly | Significantly | Significantly | Significantly |
| different | different | different | different | different |

| | | | |
|---|---|---|---|
| Stress at | Work at | | |
| 25% | 25% | | |
| elongation | elongation | Yield stress | Total work |
| [N/μm²] | [J] | [N/μm²] | [J] |

Reference 2x damaged by cold wave

| | | | |
|---|---|---|---|
| 3.21E−05 | 7.85E−04 | 1.15E−04 | 3.58E−03 |

Example according to the invention 2x with cold wave with
the addition of 1% product from synthesis example 3

| | | | |
|---|---|---|---|
| 3.64E−05 | 8.83E−04 | 1.27E−04 | 4.11E−03 |

t-Test, bilateral, pairwise

| | | | |
|---|---|---|---|
| Significantly | Significantly | Significantly | Significantly |
| different | different | different | different |

Result: by adding the product from synthesis example 3, a significant improvement in the hair structure compared to the reference was established. A significant increase in the modulus of elasticity, in the gradient, in the stress values and in the work values was observed.

3.10 Results in a Standard Commercial Cold Wave with 1% Product From Synthesis Example 4, Stage I:

Reference example: 2× permanently waved with a standard commercial permanent wave: as described under 3.2 steps f), g), c); repetition of steps f), g), c); then d) and e).

Comparative example: 2× permanently waved hair with 1% product from synthesis example 4, stage I in the cold wave, as described under 3.2: steps f) with 1% product from synthesis example 4, stage I, g), c); repetition of steps f) with 1% product from synthesis example 4, stage I, g), c); then d) and e).

| | | | | | |
|---|---|---|---|---|---|
| Hair cross | | | Stress | Stress at | Work at |
| section | Modulus of | Elastic | plateau | 15% | 15% |
| area | elasticity | gradient | range | elongation | elongation |
| [μm²] | [N/m²] | [N/mm] | [N/μm²] | [N/μm²] | [J] |

Reference

| | | | | | |
|---|---|---|---|---|---|
| 4.82E+03 | 7.11E+08 | 1.10E−01 | 2.57E−05 | 2.43E−05 | 4.27E−04 |

Permanent wave + 1% product from synthesis example 4, stage I

| | | | | | |
|---|---|---|---|---|---|
| 4.64E+03 | 8.05E+08 | 1.20E−01 | 2.76E−05 | 2.67E−05 | 4.62E−04 |

t-Test, bilateral, pairwise

| | | | | | |
|---|---|---|---|---|---|
| Signifi- | Significantly | Signifi- | Signifi- | Signifi- | Signifi- |
| cantly | different | cantly | cantly | cantly | cantly |
| different | | different | different | different | different |

| | | |
|---|---|---|
| Stress at | Work at | |
| 25% | 25% | Elongation |
| elongation | elongation | at break |
| [N/μm²] | [J] | [%] |

Reference

| | | |
|---|---|---|
| 3.11E−05 | 8.20E−04 | 5.59E+01 |

Permanent wave + 1% product from
synthesis example 4, stage I

| | | |
|---|---|---|
| 3.32E−05 | 8.74E−04 | 5.49E+01 |

t-Test, bilateral, pairwise

| | | |
|---|---|---|
| Significantly | Significantly | Significantly |
| different | different | different |

Result: by applying the permanent wave with 1% product from synthesis example 4, stage I, significant strengthening of the hair compared with the reference is found. The stress values and work values, and also the gradient and the modulus of elasticity are significantly increased. The hair cross section area and the elongation at break are significantly smaller.

3.11 Results in a Standard Commercial Cold Wave with 1% Product From Synthesis Example 4, Stage II:

Reference example: 2× permanently waved with a standard commercial permanent wave: as described under 3.2 steps f), g), c); repetition of steps f), g), c); then d) and e).

Comparative example: 2× permanently waved hair with 1% product from synthesis example 4, stage II in the cold wave, as described under 3.2: steps f), with 1% product from synthesis example 4, stage II, g), c); repetition of steps f) with 1% product from synthesis example 4, stage II, g), c); then d) and e).

| | | | | | |
|---|---|---|---|---|---|
| | | Stress | Stress at | Work at | Work at |
| Modulus of | Elastic | plateau | 15% | 15% | 25% |
| elasticity | gradient | range | elongation | elongation | elongation |
| [N/m²] | [N/mm] | [N/μm²] | [N/μm²] | [J] | [J] |

Reference

| | | | | | |
|---|---|---|---|---|---|
| 7.84E+08 | 1.12E−01 | 2.76E−05 | 2.65E−05 | 4.32E−04 | 8.24E−04 |

Permanent wave + 1% product from synthesis example 4, stage II

| | | | | | |
|---|---|---|---|---|---|
| 8.60E+08 | 1.22E−01 | 2.90E−05 | 2.78E−05 | 4.52E−04 | 8.53E−04 |

t-Test, bilateral, pairwise

| | | | | | |
|---|---|---|---|---|---|
| Significantly | Signifi- | Signifi- | Signifi- | Signifi- | Signifi- |
| different | cantly | cantly | cantly | cantly | cantly |
| | different | different | different | different | different |

Result: for the permanent wave with 1% product from synthesis example 4, stage II, a positive effect is found, since the stress values and the work values, and also the gradient and the modulus of elasticity increase significantly.

The comparison of the results which have been achieved using the polysulfide according to the invention (i.e. the products from the synthesis examples according to the invention) with those using the comparison material (product from synthesis example 4, stage II) shows that the polysulfide according to the invention more effectively improves the hair structure: cf. the table below.

| | Improvement¹⁾ through treatment with | |
|---|---|---|
| | polysulfide | |
| Parameter | according to the invention | comparison material |
| Modulus of elasticity | 18.4% | 9.7% |
| Gradient | 16.2% | 8.9% |
| Plateau stress | 13.4% | 5.1% |
| Elongation at break | 9% | 0% |

¹⁾Extent of improvement = change in the particular parameter compared to the blank value (= hair damaged by cold wave, i.e. analogous treatment without polysulfide according to the invention or comparison material)

3.12 Results of the sensory properties in the case of multiple treatment with the product from synthesis example 1 in the sensory assessment.

### Preparation of the Hair:

Hair tresses of the type 6634 from Alkinco weighing 2 g and 18 cm in length were used. Before the first treatment, all of the hairs were firstly cleaned for 30 min with a 12% strength Texapon NSO solution (pH 6.5), rinsed for 5 min and dried.

Treatment of the Hair in Each Case 1×:

1% product from synthesis example 1 was formulated into a cold wave pH 8.4 and the hair was treated therewith. The comparison used was a cold wave pH 8.4. The cold wave was applied to the dry hair.

### Cold Wave:

Amount: 5 g/g of hair corresponding to 10 g/tress
Contact time: 30 min
Rinsing time: 10 min with 38° C.-hot water using a hand-held shower

### Neutralization:

Amount: 5 g/g of hair corresponding to 10 g/tress
Contact time: 10 min
Rinsing time: 10 min with 38° C.-hot water using a hand-held shower
The hair was then dried overnight suspended in a climatic box.

### Solutions Used:

1. Cold wave: 1% product from synthesis example 1; 7% TGA, 0.5% Turpinal SL, 3.5% (NH₄)₂CO₃, pH 8.4
2. Reference cold wave: 7% TGA, 0.3% Turpinal SL, 3.5% (NH₄)₂CO₃, pH 8.4
3. Neutralizer: 2% H₂O₂, 1% Turpinal SL, pH 4.0

### Carrying Out the Sensory Assessment:

In order to ensure a defined environment, the tests were carried out in a climatic room at 23° C. and about 40% relative atmospheric humidity with standard daylight illumination. A panelist number of 11 was chosen. Each panelist was given new hair tresses. The test tresses were tested sensorily in pairwise comparison by all panelists in the same way.

### Evaluation:

The panelists assessed the individual parameters on a quantitative evaluation scale with appropriate anchor points at both ends on which they positioned both products according to the perceived difference. The distances thus obtained are a measure of the extent of the difference.

The statistical safeguard was carried out with the t-test for paired samples.
Results after 2 Treatments:

(D) cold wave+1% product from synthesis example 1 against (J) cold wave as reference.

| | | Mean | | |
|---|---|---|---|---|
| Attribute | Code | value | P values | Assessment |
| Volume before combing | D | 29.4 | 0.701 | |
| little - much | J | 31.5 | | |
| Straggliness before combing | D | 49.7 | 0.986 | |
| not straggly - straggly | J | 49.6 | | |
| Shine before combing | D | 45.3 | 0.850 | |
| matt - shiny | J | 43.6 | | |
| Combability 1x | D | 74.6 | 0.173 | |
| difficult - easy | J | 66.8 | | |
| Combability 5x | D | 70.2 | 0.712 | |
| difficult - easy | J | 72.9 | | |
| Charging | D | 64.5 | 0.987 | |
| none - much | J | 64.7 | | |
| Volume after combing | D | 38.0 | 0.081 | T |
| little - much | J | 31.7 | | |
| Straggliness after combing | D | 19.7 | 0.291 | |
| not straggly - straggly | J | 17.3 | | |
| Shine after combing | D | 55.8 | 0.580 | |
| matt - shiny | J | 52.5 | | |
| Smoothness in the hair overall | D | 62.0 | 0.015 | HS |
| rough - smooth | J | 75.6 | | |
| Smoothness at the ends | D | 54.6 | 0.170 | |
| rough - smooth | J | 61.5 | | |
| Noise upon wiping | D | 21.0 | 0.355 | |
| soft - loud | J | 25.3 | | |
| Softness in the hair overall | D | 54.8 | 0.239 | |
| hard - soft | J | 62.8 | | |
| Softness at the ends | D | 60.1 | 0.831 | |
| hard - soft | J | 58.7 | | |
| Thickness of the hair | D | 56.7 | 0.601 | |
| thin - thick | J | 53.0 | | |
| Residues in the hair | D | 26.1 | 0.369 | |
| none - many | J | 35.1 | | |
| Residues on the skin | D | 22.6 | 0.257 | |
| none - many | J | 27.7 | | |
| Stiffness | D | 46.4 | 0.100 | |
| flexible - stiff | J | 36.3 | | |
| Stylability | D | 55.9 | 0.910 | |
| not stylable - stylable | J | 54.9 | | |

Results after 3 treatments: (D) cold wave + 1% product from synthesis example 1 against (J) cold wave as reference

| | | Mean | | |
|---|---|---|---|---|
| Attribute | Code | value | P values | Assessment |
| Volume before combing | D | 22.6 | 0.125 | |
| little - much | J | 16.3 | | |
| Straggliness before combing | D | 52.9 | 0.141 | |
| not straggly - straggly | J | 59.1 | | |
| Shine before combing | D | 56.2 | 0.458 | |
| matt - shiny | J | 51.0 | | |
| Combability 1x | D | 72.5 | 0.454 | |
| difficult - easy | J | 67.8 | | |
| Combability 5x | D | 76.0 | 0.181 | |
| difficult - easy | J | 85.2 | | |
| Charging | D | 68.4 | 0.236 | |
| none - much | J | 79.7 | | |
| Volume after combing | D | 47.0 | 0.178 | |
| little - much | J | 40.3 | | |
| Straggliness after combing | D | 18.6 | 0.086 | T |
| not straggly - straggly | J | 25.0 | | |
| Shine after combing | D | 57.7 | 0.744 | |
| matt - shiny | J | 55.5 | | |
| Smoothness in the hair overall | D | 61.9 | 0.732 | |
| rough - smooth | J | 64.5 | | |
| Smoothness at the ends | D | 59.7 | 0.343 | |
| rough - smooth | J | 65.1 | | |
| Noise upon wiping | D | 32.1 | 0.759 | |
| soft - loud | J | 34.0 | | |
| Softness in the hair overall | D | 64.1 | 0.560 | |
| hard - soft | J | 60.8 | | |
| Softness at the ends | D | 59.1 | 0.914 | |
| hard - soft | J | 59.7 | | |
| Thickness of the hair | D | 50.8 | 0.474 | |
| thin - thick | J | 47.6 | | |
| Residues in the hair | D | 14.8 | 0.741 | |
| none - many | J | 13.1 | | |
| Residues on the skin | D | 13.9 | 0.199 | |
| none - many | J | 18.5 | | |
| Stiffness | D | 34.1 | 0.922 | |
| flexible - stiff | J | 33.5 | | |
| Stylability | D | 44.1 | 0.643 | |
| not stylable - stylable | J | 40.4 | | |

Results after 4 treatments: (D) cold wave + 1% product from synthesis example 1 against (J) cold wave as reference

| | | Mean | | |
|---|---|---|---|---|
| Attribute | Code | value | P values | Assessment |
| Volume before combing | D | 12.0 | 0.040 | S |
| little - much | J | 7.6 | | |
| Straggliness before combing | D | 54.7 | 0.171 | |
| not straggly - straggly | J | 58.7 | | |
| Shine before combing | D | 55.8 | 0.196 | |
| matt - shiny | J | 47.9 | | |
| Combability 1x | D | 80.5 | 1.000 | |
| difficult - easy | J | 80.5 | | |
| Combability 5x | D | 81.6 | 0.387 | |
| difficult - easy | J | 86.4 | | |
| Charging | D | 55.1 | 0.048 | S |
| none - much | J | 68.6 | | |
| Volume after combing | D | 39.2 | 0.182 | |
| little - much | J | 35.7 | | |
| Straggliness after combing | D | 22.2 | 0.546 | |
| not straggly - straggly | J | 23.8 | | |
| Shine after combing | D | 61.9 | 0.063 | T |
| matt - shiny | J | 53.9 | | |
| Smoothness in the hair overall | D | 72.9 | 0.636 | |
| rough - smooth | J | 70.5 | | |
| Smoothness at the ends | D | 76.1 | 0.651 | |
| rough - smooth | J | 73.8 | | |
| Noise upon wiping | D | 30.7 | 0.771 | |
| soft - loud | J | 32.2 | | |
| Softness in the hair overall | D | 65.1 | 0.951 | |
| hard - soft | J | 65.6 | | |
| Softness at the ends | D | 64.0 | 0.918 | |
| hard - soft | J | 64.6 | | |
| Thickness of the hair | D | 59.2 | 0.263 | |
| thin - thick | J | 52.5 | | |
| Residues in the hair | D | 13.8 | 0.415 | |
| none - many | J | 9.5 | | |
| Residues on the skin | D | 14.1 | 0.620 | |
| none - many | J | 13.2 | | |
| Stiffness | D | 40.8 | 0.512 | |
| flexible - stiff | J | 37.0 | | |
| Stylability | D | 40.1 | 0.856 | |
| not stylable - stylable | J | 41.5 | | |

The p values are to be interpreted as follows:
p<0.01 products differ with high significance with regard to this attribute (HS)
0.01<p<0.05 products differ significantly with regard to this attribute (S)
0.01<p<0.05 products differ significantly with regard to this attribute (S)
0.05<p<0.1 products do not differ significantly with regard to this attribute although a trend in the product difference tendency with regard to this attribute can be seen (T)
p>0.1 products do not differ significantly with regard to this attribute, no similar results, but no statement with regard to significance possible.

The attributes differ in the direction of the markedness which is emphasized in the tables by underlining.

### Result:

2 treatments: after 2 treatments, the hair treated with 1% product from synthesis example 1 felt highly significantly less smooth and had a tendency to have more volume after combing.

3 treatments: the hair treated with 1% product from synthesis example 1 had a tendency to be less straggly than the reference permanent wave.

4 treatments: prior to combing, a significantly greater volume was established as result of the treatment with 1% product from synthesis example 1. Likewise, the hair tresses exhibited significantly less charging than the reference. The hair treated with 1% product from synthesis example 1 had a tendency to shine more after combing.

### Summary Assessment:

In the course of the treatments, only positive effects in favor of the treatment according to the invention were found. After 2 and 4 applications, a volume increase was established, the straggliness and the charging were likewise reduced. The shine was likewise improved. The reduction in smoothness observed after 2 treatments no longer arose after further applications.

### Formulation Examples

Example Formulation 1: Waving Composition

7% TGA, 0.3% Turpinal SL, 1% polysulfide according to the invention¹⁾, 3.5% (NH₄)₂CO₃, pH 8.4

Example Formulation 2: Waving Composition

12% ammonium thioglycolate, 5% NH₄HCO₃, 0.5% NH₃, 1% Cremophor RH 40²⁾, 1% Lamepon S³⁾, 1% polysulfide according to the invention¹⁾, perfume 0.5%

Example Formulation 3: 2-Phase 2-Component Permanent Wave

| | |
|---|---|
| a) | Base waving lotion |

| | | |
|---|---|---|
| aa) | aqueous phase | |
| | ammonia 25% | 0.9 |
| | polysulfide according to the invention¹⁾ | 1 |
| | Merquat 100⁴⁾ | 0.1 |
| | ammonium hydrogencarbonate | 5.5 |
| | water | ad 100 |
| bb) | oil phase | |
| | perfume | 10 |
| | C.I. 61565 | 0.002 |
| | Dow Corning 345⁵⁾ | ad 100 |

| | |
|---|---|
| b) | Activator |

| | |
|---|---|
| ammonium thiolactate 70% | 21 |
| ammonium thioglycolate 71% | 57 |
| Turpinal SL | 1 |
| Eumulgin L⁶⁾ | 4 |
| water | ad 100 |

57.5 ml of base waving lotion consisting of 52 ml of aqueous phase and 5.5 ml of oil phase, and 22.5 ml of activator are mixed prior to application and give 80 ml of a ready-to-use permanent wave.

Example Formulation 4: Alkaline Pre- and/or Interim Treatment

| | |
|---|---|
| Dow Corning 939⁷⁾ | 2 |
| ethanol 96% | 32 |
| phytantriol | 0.1 |
| Merquat 100 | 0.1 |
| monoethanolamine | 0.5 |
| polysulfide according to invention¹⁾ | 1 |
| water | ad 100 |

Example Formulation 5: Alkaline, Reductive Pre- and/or Interim Treatment

| | |
|---|---|
| Dow Corning 939 | 2 |
| ethanol 96% DEP denatured | 32 |
| phytantriol | 0.1 |
| Merquat 100 | 0.1 |
| ammonia 25% | 0.5 |
| ammonium thioglycolate 71% | 3 |
| polysulfide according to invention¹⁾ | 1 |
| water | ad 100 |

Example Formulation 6: Ready-to-Use Permanent Wave for Normal Hair

| | |
|---|---|
| ammonium thioglycolate 71% | 18 |
| Turpinal SL | 0.3 |
| ammonia 25% | 2 |
| ammonium hydrogencarbonate | 8 |
| Cremophor RH 40 | 1 |
| Lamepon S | 1 |
| perfume | 0.5 |
| Gluadin WQ⁸⁾ | 0.1 |
| Merquat 100 | 0.1 |
| polysulfide according to invention¹⁾ | 0.5 |
| water | ad 100 |

Example Formulation 7: Ready-to-Use Permanent Wave for Colored Hair

| | |
|---|---|
| ammonium thioglycolate 71% | 11 |
| Turpinal SL | 0.3 |
| ammonia 25% | 2 |
| ammonium hydrogencarbonate | 4 |
| Cremophor RH 40 | 1 |
| Lamepon S | 1 |
| perfume | 0.5 |
| Gluadin WQ | 0.2 |
| Merquat 100 | 0.2 |
| polysulfide according to invention¹⁾ | 1 |
| water | ad 100 |

¹⁾polysulfide according to the invention = for example product from one of synthesis examples 1 to 3
²⁾hydrogenated castor oil + 40-45 ethylene oxide (INCI name: PEG-40 Hydrogenated Castor Oil) (BASF)
³⁾protein coconut fatty acid condensate, potassium salt (INCI name: Potassium Cocoyl Hydrolyzed Collagen) active substance content about 32% (COGNIS)
⁴⁾poly(dimethyldiallylammonium chloride) (40% active substance, INCI name Polyquaternium-6, CHEMVIRON)
⁵⁾INCI name Cyclomethicone (Dow Corning)
⁶⁾2-hydroxy fatty alcohol alkoxylate, INCI name PPG-2-Ceteareth-9 (COGNIS)
⁷⁾INCI name Amodimethicone (Dow Corning)
⁸⁾INCI name Laurdimonium Hydroxypropyl Hydrolyzed Wheat Protein (COGNIS)

1. A method for restructuring keratin fibers, in which a keratin fiber is brought into contact
(a) with an aqueous preparation of at least one polysulfide of the formula (I) 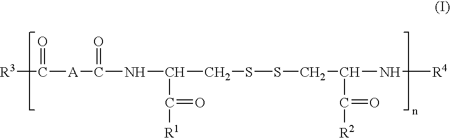
in which
R³ is hydroxy or the radical 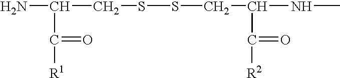
R⁴ is hydrogen or the radical 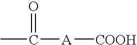
A is a bond or a divalent saturated or a mono- or polyunsaturated aliphatic or aromatic hydrocarbon radical having 1 to 20 carbon atoms, which may be substituted by one or more halogen, hydroxy or carboxy groups and where the shortest bond between the two carbonyl groups adjacent to group A consists of up to 12 carbon atoms, and in which
R¹ and R² may be identical or different and are chosen from
R⁵O— and 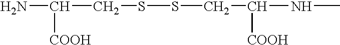
where R⁵ is hydrogen or an alkyl group having 1 to 6 carbon atoms,
n is an integer from 1 to 100,
and where one or more carboxyl group(s) may be in the form of one or more of its/their salt(s),
and a reducing agent,
simultaneously or successively, and then
(b) with an oxidizing agent.

2. The method as claimed in claim 1, characterized in that the keratin fiber is a hair.

3. The method as claimed in claim 1 or 2, characterized in that the restructuring involves fiber strengthening.

4. The method as claimed in one of claims 1 to 3, characterized in that, in formula (I), A is a divalent saturated aliphatic hydrocarbon radical having 2 to 6 carbon atoms.

5. The method as claimed in claim 4, characterized in that, in formula (I), A is the ethane-1,2-diyl radical.

6. The method as claimed in one of claims 1 to 5, characterized in that, in formula (I), n is an integer from 1 to 10.

7. The method as claimed in one of claims 1 to 6, characterized in that the reducing agent is a keratin-reducing compound which is chosen from thioglycolic acid, thiolactic acid, thiomalic acid, phenylthioglycolic acid, mercaptoethanesulfonic acid, and salts and esters thereof (such as, for example, isooctyl thioglycolate and isopropyl thioglycolate), cysteamine, cysteine, Bunte salts and salts of sulfurous acid, alkali metal disulfites, such as, for example, sodium disulfite (Na₂S₂O₅) and potassium disulfite (K₂S₂O₅), and magnesium disulfite and ammonium disulfite ((NH₄)₂S₂O₅), hydrogensulfites as alkali metal, magnesium, ammonium or alkanolammonium salts based on a C₂-C₄-mono-, di- or trialkanolamine, and sulfites as alkali metal, ammonium or alkanolammonium salts based on a C₂-C₄-mono-, di- or trialkanolamine.

8. The method as claimed in claim 7, characterized in that the reducing agent is chosen from thioglycolic acid and thiolactic acid, and salts thereof.

9. The method as claimed in one of claims 1 to 8, characterized in that the oxidizing agent is chosen from oxygen, air, H₂O₂, disulfides, sodium perborate and hydrates thereof, sodium and potassium bromate, sodium chlorite, sodium or potassium persulfate, sodium iodate, calcium or magnesium bromate, tetrathionates, glyoxal, glutaraldehyde, and mixtures of these substances.

10. The method as claimed in claim 9, characterized in that the oxidizing agent is chosen from air and/or H₂O₂.

11. The method as claimed in one of claims 1 to 10, characterized in that the reducing agent used is thioglycolic acid and/or thiolactic acid or one of its salts, and the oxidizing agent used is H₂O₂.

12. The method as claimed in one of claims 1 to 11, characterized in that the preparation comprises in total 0.01 to 5% by weight, but in particular 0.1 to 2% by weight, of one or more polysulfides of the formula (I).

13. The method as claimed in one of claims 1 to 12, characterized in that the preparation has a pH between 7 and 9.5.

14. The method as claimed in one of claims 1 to 13, characterized in that step (a) of the method is carried out in the presence of cystine.

15. The method as claimed in claim 14, characterized in that cystine, based on the weight of the at least one polysulfide of the formula (I), is present in an amount of from 5 to 1000% by weight, but in particular 50 to 500% by weight.

16. The method as claimed in one of claims 1 to 15, characterized in that, in step (a) of the method, the fiber is brought into contact simultaneously with the polysulfide of the formula (I) and the reducing agent.

17. The method as claimed in one of claims 1 to 16, wherein, in step (a) of the method, the fiber remains in contact with the polysulfide of the formula (I) and the reducing agent for a contact time from 1 to 60 minutes, but in particular from 5 to 30 minutes.

18. The method as claimed in one of claims 1 to 17, characterized in that it serves for the permanent shaping of keratin fibers, and in particular is a permanent waving method for human hair.

19. An aqueous preparation for use in a method as claimed in one of claims 1 to 18, comprising in total 0.01 to 5% by weight, but in particular 0.1 to 2% by weight, of one or more polysulfides of the formula (I).

20. The preparation as claimed in claim 19, characterized in that a buffer system with a pH of from 7 to 9.5 is present.

21. The preparation as claimed in claim 19 or 20, characterized in that 0.001 to 10% by weight, but in particular 0.05 to 5% by weight, of cystine are present.

22. The preparation as claimed in one of claims 19 to 21, characterized in that a keratin-reducing compound, in particular thioglycolic acid and/or thiolactic acid or a salt thereof is present.

23. A kit for use in a method as claimed in one of claims 1 to 18, comprising
(a) a preparation as claimed in one of claims 19 to 21 and
(b) a preparation comprising a reducing agent.

24. The kit as claimed in claim 23, characterized in that it further includes a preparation comprising an oxidizing agent.

25. A kit for use in a method as claimed in one of claims 1 to 18, comprising
(a) a preparation as claimed in claim 22 and
(b) a preparation comprising an oxidizing agent.

26. A fiber, in particular keratin fiber, obtainable by a method as claimed in one of claims 1 to 18.

27. The use of a polysulfide of the formula (I), as defined in claim 1, for restructuring keratin fibers, in particular hair.

28. The use as claimed in claim 27, characterized in that the restructuring includes fiber strengthening.
